(19) United States
(12) Patent Application Publication
(10) Pub. No.: US 2026/0364165 A1
(43) Pub. Date: Nov. 19, 2026

(54) VACCINE MIXING METHOD, SYRINGE AND SYSTEM

(71) Applicant: BioNTech SE, Mainz (DE)

(72) Inventors: Alexander Muik, Seeheim-Jugenheim (DE); Ugur Sahin, Mainz (DE)

(21) Appl. No.: 19/158,734

(22) PCT Filed: Apr. 12, 2024
(86) PCT No.: PCT/EP24/60037

§ 371 Date: Aug. 21, 2025

Related U.S. Application Data
(60) Provisional application No. 63/459,542, filed on Apr. 14, 2023.

(51) Int. Cl. A61K 39/215; A61J 1/20; A61K 39/00; A61K 39/12; A61K 39/145

(52) U.S. Cl. CPC A61K 39/215; A61J 1/2048; A61K 39/12; A61K 39/145; A61K 2039/5252; A61K 2039/53; A61K 2039/70

(57) ABSTRACT

A method of preparing a combination vaccine includes: (a) obtaining a first amount of a first vaccine in a first syringe, (b) obtaining a second amount of a second vaccine In a second syringe, (c) connecting the first and the second syringe using an adapter, (d) mixing the first vaccine and the second vaccine, and (e) transferring the mixture of the first vaccine and the second vaccine, as needed, to the first syringe or the second syringe, and discarding the syringe that the mixture was not transferred to.

### CROSS-REFERENCE TO RELATED APPLICATIONS

This application claims priority to U.S. Provisional Patent Application No. 63/459,542, filed Apr. 14, 2023; the title of which is “COMBINATION VACCINES,” and the content of which is incorporated herein by reference in its entirety.

### BACKGROUND

Due to ongoing Covid-19 (i.e., Sars-CoV-2) outbreaks as well as seasonally prevalent influenza outbreaks (among outbreaks of other types of diseases), it is often inconvenient and/or time-consuming to schedule and receive two or more separate vaccines, especially during rapid and/or severe outbreaks.

### SUMMARY

Among other things, in some embodiments, the present disclosure identifies the source of one or more challenges that can be associated with having to administer multiple vaccines within a short period of time. Current drug (e.g., vaccine) delivery systems may include different types of delivery modalities and payloads. For example, SARS-CoV-2 (i.e., COVID-19) vaccines may include an RNA molecule (for example, mRNA) formulated in a lipid nanoparticle (LNP) delivery system while influenza (i.e., flu) vaccines may include one or more polypeptide antigens and/or inactivated viral particles or portions thereof. In some instances, the different delivery modalities can interact with one another when coformulated, resulting in mitigation of the immunogenicity of one or more of the vaccines. Accordingly, it is typical for vaccines to be developed, transported, and administered individually. In some embodiments, the present disclosure provides methods for producing a combined vaccine, e.g., comprising mixing two or more vaccines. In some embodiments, methods described herein can provide certain advantages as compared to previous methods for producing combined vaccines (e.g., advantages as compared to methods that comprise mixing two or more vaccines in a vial).

Administering vaccines individually, can be disadvantageous because, among other issues, it Increases the number of shots that a subject receives, which can decrease patient compliance. Given the number of subjects vaccinated each year, increased patient compliance can result in millions of fewer people becoming sick and thousands of lives being saved every year, especially for diseases that are prevalent in the population. For example, the CDC estimates that, between 2010 and 2023, flu alone caused 9.3-41 million illnesses, 100,000-730,000 hospitalizations, and 4,900-51,000 deaths every year. See https://covid.cdc.gov/covid-data-tracker/#datatracker-home. Similarly, the CDC estimates that COVID-19 has resulted in over 1 million deaths between 2020 and 2023. See https://www.cdc.gov/nchs/nvss/vsrr/covid_weekly/index.htm. Thus, approaches that better enable co-administration of vaccines and increased patient compliance have the potential to avoid a huge number of Illnesses and deaths every year and provide a major benefit to society.

The present application recognizes, inter alia, strategies for avoiding potential immune interference between co-administered vaccines, thus better enabling co-administering of vaccines. Among other things, the present application recognizes that, in some embodiments, vaccines comprising different delivery modalities and/or payloads can interfere with each because of interactions between nonhydrophobic components. In particular, vaccines comprising a lipid particle (e.g., an LNP) can interact with vaccines comprising alternative lipid particles (e.g., a viral particle comprising components of a lipid membrane), which can cause degradation of the lipid particles (e.g., LNP), thus resulting in a decreased immune response by one or both of the vaccine components.

In some embodiments, methods described herein that comprise mixing two or more vaccines in a syringe can be more easily performed as compared to previous methods (e.g., as compared to methods that comprise mixing two or more vaccines in a vial). For example, in some embodiments, methods described herein can comprise fewer steps; take less time; be less prone to error; and/or require less training, skill, or experience to perform as compared to previous methods.

In some embodiments, methods described herein can be more versatile and/or more readily adaptable to combining many different types of vaccines as compared to previous methods. For example, in some embodiments, methods described herein do not require specially-made equipment (e.g., can be performed using items commonly available to an administering health care professional; can be performed in a variety of settings; and/or can be performed by healthcare professionals having a range of skill levels/training). For example, in some embodiments, methods described herein can be compatible with standard syringes. In some embodiments, methods described herein can be performed in a variety of settings (including, e.g., a pharmacy or a hospital).

In some embodiments, methods described herein can reduce volume loss as compared to other methods (e.g., as compared to methods that comprise mixing in a vial).

In some embodiments, methods described herein produce a sterile combination vaccine (e.g., in some embodiments, methods described herein that use a sterile single-packed adapter can produce a sterile combination vaccine).

In some embodiments, methods disclosed herein can be used to formulate and administer a combination vaccine with significantly less quality control testing, regulatory review, and/or stability concerns as compared to a standard co-formulation vaccine (e.g., as compared to a co-formulation prepared by a manufacturer).

In some embodiments, the present disclosure provides a method of preparing a combination vaccine, wherein the method comprises:
(a) obtaining a first amount of a first vaccine in a first syringe,
(b) obtaining a second amount of a second vaccine in a second syringe,
(c) connecting the first syringe and the second syringe using an adapter,
(d) transferring the first vaccine to the second syringe, and
(e) discarding the first syringe.

In some embodiments, a first vaccine and a second vaccine used in a method described herein are not stable when coformulated (e.g., wherein the immunogenicity of the first vaccine and/or the second vaccine decreases after mixing and storing at 4 °C or room temperature for 1 week or longer, 1 day or longer, or 1 hour or longer as compared to the first vaccine and/or the second vaccine stored under similar (e.g., the same) conditions). In some embodiments, a first vaccine comprises a first delivery modality, wherein the first delivery modality comprises a lipid nanoparticles (LNP), a lipoplex (LPX), a liposome, and/or an oligosaccharide, and a second vaccine comprises:
(i) an inactivated virus, a recombinant polypeptide, a live attenuated virus, a subunit vaccine, or a split vaccine;
(ii) a second delivery modality, wherein the second delivery modality comprises a lipid nanoparticle (LNP), a lipoplex, a liposome, and/or a lipophilic oligosaccharide, and wherein the second delivery modality is different from the first delivery modality; and/or
(iii) a recombinant polypeptide.

In some embodiments, a method provided herein uses a second vaccine comprising a recombinant polypeptide comprising a lipophilic region that interacts with the first delivery modality. In some embodiments, a second vaccine comprises a recombinant polypeptide and a lipid.

In some embodiments, a method comprises mixing a first vaccine and a second vaccine prior to transferring the first vaccine to the second syringe, by repeatedly transferring liquid from the first syringe to the second syringe (e.g., until the first vaccine and the second vaccine form a substantially homogenous mixture).

In some embodiments, a method comprises transferring a first vaccine to a second syringe by a method comprising compressing the plunger of a first syringe only a single time, so that the first vaccine and a second vaccine form a substantially heterogenous mixture.

In some embodiments, a first vaccine and/or a second vaccine used in a method are a nucleic acid vaccine.

In some embodiments, a nucleic acid vaccine is an RNA vaccine (e.g., an LNP-formulated mRNA).

In some embodiments, a second vaccine in a method comprises (i) one or more of an inactivated virus, a recombinant polypeptide, or a live attenuated virus; and/or (ii) comprises a non-adjuvanted vaccine, an adjuvanted vaccine, a subunit vaccine, or a split vaccine.

In some embodiments, a first vaccine in a method is an RNA vaccine (e.g., comprises LNP-formulated mRNA) and a second vaccine in a method comprises an inactivated virus.

In some embodiments, a first vaccine in a method is an RNA vaccine (e.g., comprises LNP-formulated mRNA) and a second vaccine in a method comprises a recombinant polypeptide (optionally with a lipophilic adjuvant).

In some embodiments, a first vaccine and a second vaccine each deliver one or more viral antigens (e.g., wherein the first vaccine and the second vaccine each deliver one or more antigens associated with a different virus).

In some embodiments, a first vaccine and a second vaccine each deliver one or more antigens associated with a respiratory virus (e.g., wherein the first vaccine and the second vaccine each deliver one or more antigens associated with a different respiratory virus).

In some embodiments, a first vaccine and/or a second vaccine are each a seasonally-updated vaccine (e.g., a monovalent, bivalent, trivalent, or quadrivalent seasonally-updated vaccine), optionally wherein the first vaccine and the second vaccine are updated at a similar or the same frequency.

In some embodiments, a first vaccine is a SARS-CoV-2 vaccine.

In some embodiments, a SARS-CoV-2 vaccine is a commercially available vaccine and/or a vaccine that has been approved by a government regulatory authority (e.g., the US FDA and/or the EMA).

In some embodiments, a SARS-CoV-2 vaccine is an mRNA vaccine.

In some embodiments, a SARS-CoV-2 vaccine comprises mRNA-1273, Ad26.CoV2.S, ChAdxOx1, NVX-CoV2373, CvnCoV, GAM-COVID0Vac, CoronaVac, BBIBP-CorV, Ad5-nCoV, zf2001, SCB-2019, JNJ 78436735, or BNT162b2.

In some embodiments, a second vaccine is an influenza vaccine.

In some embodiments, an influenza vaccine is an inactivated influenza virus (e.g., Fluzone®, Fluzone high-dose quadrivalent®, Fluzone quadrivalent®, Fluzone intradermal quadrivalent®, Fluzone quadrivalent southern hemisphere®, Fluad®, Fluad quadrivalent®, Afluria Quadrivalent®, Fluarix Quadrivalent®, FluLaval Quadrivalent®, or Flucelvax Quadrivalent®), a recombinant influenza vaccine (e.g., Flublok quadrivalent®), a live attenuated influenza vaccine (e.g., FluMist Quadrivalent®), an unadjuvanted influenza vaccine, an adjuvant influenza vaccine, or a subunit or split vaccine.

In some embodiments, a second vaccine is an RSV vaccine.

In some embodiments, a first vaccine is a SARS-CoV-2 vaccine and a second vaccine is an influenza vaccine.

In some embodiments, a first vaccine is a SARS-CoV-2 vaccine and the second vaccine is an RSV vaccine.

In some embodiments, a first vaccine is BNT162b2 and a second vaccine is Fluzone®, Fluzone high-dose quadrivalent®, Fluzone quadrivalent®, Fluzone intradermal quadrivalent®, or Fluzone quadrivalent southern hemisphere®.

In some embodiments, a needle is attached to each of a first syringe and a second syringe when obtaining a first vaccine and a second vaccine, and each of the needles is removed prior to attaching the adapter.

In some embodiments, methods described herein comprise a step of administering the mixture of the first vaccine and the second vaccine to a subject.

In some embodiments, a method is performed under conditions such that there is no substantial volume loss of a first vaccine or a second vaccine (e.g., wherein the sum of the first amount of the first vaccine and the second amount of the second vaccine is approximately the same as the mixture administered to the subject, e.g., wherein the sum of the first amount of the first vaccine and the second amount of the second vaccine is within at least about 5%, at least about 4%, at least about 3%, at least about 2% or at least about 1% of the amount administered to the subject).

In some embodiments, a method described herein is performed in a pharmacy.

In some embodiments, a mixture of a first vaccine and a second vaccine is administered to a subject shortly after producing the mixture (e.g., within about 1 hour, within about 30 min, within about 15 min, within about 10 min, or within about 5 min, or within about 1 min of producing the mixture).

In some embodiments, a vaccine is administered in a hospital.

In some embodiments, one or both of a first vaccine and a second vaccine in a method are obtained from a multidose vial.

In some embodiments, In a method, (i) a first amount of a first vaccine and a second amount of a second vaccine each correspond to an amount that has been shown to provide a clinical benefit and/or prophylaxis in subjects when administered alone (e.g., shown in clinical trials to provide a clinical benefit or prophylaxis), and/or (ii) a first amount of a first vaccine and a second amount of a second vaccine each correspond to a dose that has been approved for sale by a government regulatory authority (e.g., the US FDA or the EMA).

In some embodiments, a first amount of a first vaccine and a second amount of a second vaccine are each about 100 μl to about 1 mL in a method.

In some embodiments, the combined volume of (a) a first amount of a first vaccine and (b) a second amount of a second vaccine in a method is not more than the maximum volume of a second syringe.

In some embodiments, the combined volume of (a) a first amount of a first vaccine and (b) a second amount of a second vaccine in a method, is not more than 1 mL.

In some embodiments, a first amount of a first vaccine, and a second amount of a second vaccine in a method are each about 200 μL to about 600 μL.

In some embodiments, a first amount of a first vaccine is about 500 μL and a second amount of a second vaccine is about 300 μL in a method.

In some embodiments, a first amount of a first vaccine in a method is about 200 μL or 300 μL.

In some embodiments, a second amount of a second vaccine in a method is about 250 μL or about 500μL.

In some embodiments, in a method,
(a) a first amount of a first vaccine is about 200 μL and a second amount of a second vaccine is about 250 μL;
(b) a first amount of a first vaccine is about 200 μL and a second amount of a second vaccine is about 500 μL;
(c) a first amount of a first vaccine is about 300 μL and a second amount of a second vaccine is about 250 μL; or
(d) a first amount of a first vaccine is about 300 μL and a second amount of a second vaccine is about 500 μL.

In some embodiments, a method described herein further comprises:
(f) obtaining a third amount of a third vaccine in a third syringe,
(g) connecting the third syringe to a syringe comprising a mixture of a first vaccine and a second vaccine using an adapter,
(h) mixing the third vaccine with the mixture of the first vaccine and the second vaccine, and
(i) transferring the mixture of the first, second, and third vaccine to (i) the syringe that previously held the mixture of the first vaccine and the second vaccine or (ii) the third syringe, and discarding the syringe that the mixture was not transferred to.

In some embodiments, each of a first vaccine, a second vaccine, and a third vaccine deliver one or more antigens of a virus associated with a respiratory disease (e.g., wherein each vaccine delivers one or more antigens of a different virus associated with a respiratory disease).

In some embodiments, in a method,
(a) a first vaccine is a SARS-CoV-2 vaccine, a second vaccine is an influenza vaccine, and a third vaccine is an RSV vaccine;
(b) a first vaccine is a SARS-CoV-2 vaccine, a second vaccine is an RSV vaccine, and a third vaccine is an influenza vaccine;
(c) a first vaccine is an influenza vaccine, a second vaccine is an RSV vaccine, and a third vaccine is a SARS-CoV-2 vaccine;
(d) a first vaccine is an influenza vaccine, a second vaccine is a SARS-CoV-2 vaccine, and a third vaccine is an RSV vaccine; or
(e) a first vaccine is a SARS-CoV-2 vaccine, a second vaccine is an RSV vaccine, and a third vaccine is an influenza vaccine.

In some embodiments, at least one (e.g., all) of a first syringe, a second syringe, and, if present, a third syringe, is not a uni-directional syringe (e.g., at least one of the syringes is a bidirectional syringe and/or at least one of the syringes does not have a stopper that prevents motion of the plunger in one direction).

In some embodiments, an adapter described herein is a sterile single-packaged adapter.

In some embodiments, an adapter described herein is a Luer-Luer adapter (e.g., a Luer dual female adapter).

In some embodiments, the present disclosure provides a system comprising:
a first syringe;
an adapter comprising a first end and a second end, the first end of the adapter coupled to a distal end of the first syringe;
a second syringe coupled at a distal end to the second end of the adapter; and
multiple vaccines disposed within the first syringe and/or the second syringe, a first vaccine of the multiple vaccines being disposed within the first syringe and a second vaccine of the multiple vaccines being disposed within the second syringe.

In some embodiments, the present disclosure provides a system comprising:
a first syringe;
an adapter comprising a first end and a second end, the first end of the adapter coupled to a distal end of the first syringe;
a second syringe coupled at a distal end to the second end of the adapter;
multiple commingled vaccines disposed within the first syringe and/or the second syringe.

In some embodiments, a system described herein comprises a first syringe and a second syringe that do not comprise needles.

In some embodiments, a system described herein comprises commingled vaccines comprise a heterogeneous mixture with a first vaccine being primarily disposed within a distal end of one syringe, and a second vaccine being primarily disposed within a proximal end of the same syringe.

In some embodiments, a syringe comprises a heterogenous mixture comprising two distinct regions, a first vaccine occupying a first distinct region and a second vaccine occupying a second distinct region, wherein the first and second distinct region are visibly distinguishable.

In some embodiments, commingled vaccines comprise a homogenous mixture.

In some embodiments, the present disclosure provides a composition produced by a method described herein.

In some embodiments, a composition described herein comprises a first vaccine and a second vaccine, wherein the first vaccine is an RNA vaccine (e.g., an mRNA-LNP vaccine) and the second vaccine is a non-nucleic acid vaccine.

In some embodiments, a nucleic acid vaccine is an RNA vaccine.

In some embodiments, an RNA vaccine comprises RNA formulated as particles.

In some embodiments, particles are lipoplex particles (LPX) or lipid nanoparticles (LNP).

In some embodiments, a non-nucleic acid vaccine comprises an inactivated virus, a recombinant polypeptide, a live attenuated virus, a non-adjuvanted vaccine, an adjuvanted vaccine, a subunit vaccine or a split vaccine, or any combination thereof.

In some embodiments, a composition comprises a first vaccine that is an RNA vaccine (e.g., comprises LNP-formulated mRNA) and a second vaccine that comprises an inactivated virus.

In some embodiments, a composition comprises a first vaccine and a second vaccine that each deliver one or more viral antigens (e.g., wherein the first vaccine and the second vaccine each deliver one or more antigens associated with a different virus).

In some embodiments, a composition comprises a first vaccine and a second vaccine that each deliver one or more antigens associated with a respiratory virus (e.g., wherein the first vaccine and the second vaccine each deliver one or more antigens associated with a different respiratory virus).

In some embodiments, a composition comprises a first vaccine and/or a second vaccine that are each a seasonally updated vaccine.

In some embodiments, a composition comprises a first vaccine that comprises a SARS-CoV-2 vaccine. Suitable SARS-CoV-2 vaccines are known in the art, and include, e.g., those described in Creech, C. Buddy, Shannon C. Walker, and Robert J. Samuels. “SARS-CoV-2 vaccines,” Jama 325.13 (2021): 1318-1320; Chakraborty, Chiranjib, Manojit Bhattacharya, and Kuldeep Dhama, “SARS-CoV-2 vaccines, vaccine development technologies, and significant efforts in vaccine development during the pandemic: the lessons learned might help to fight against the next pandemic,” Vaccines 11.3 (2023): 682; and Kyriakidis, Nikolaos C., et al. “SARS-CoV-2 vaccines strategies: a comprehensive review of phase 3 candidates,” npj Vaccines 6.1 (2021): 28.

In some embodiments, a composition comprises a first vaccine that comprises a SARS-CoV-2 vaccine is a commercially approved vaccine.

In some embodiments, a SARS-CoV-2 vaccine comprises an mRNA vaccine.

In some embodiments, a SARS-CoV-2 vaccine is selected from mRNA-1273, Ad26.CoV2.S, ChAdxOx1, NVX-CoV2373, CvnCoV, GAM-COVID0Vac, CoronaVac, BBIBP-CorV, Ad5-nCoV, zf2001, SCB-2019, JNJ 78436735, and BNT162b2.

In some embodiments, a composition comprises a second vaccine that comprises an influenza vaccine.

In some embodiments, an influenza vaccine comprises an inactivated influenza virus (e.g., Fluzone®, Fluzone high-dose quadrivalent®, Fluzone quadrivalent®, Fluzone intradermal quadrivalent®, Fluzone quadrivalent southern hemisphere®, Fluad®, Fluad quadrivalent®, Afluria Quadrivalent®, Fluarix Quadrivalent®, FluLaval Quadrivalent®, or Flucelvax Quadrivalent®), a recombinant influenza vaccine (e.g., Flublok quadrivalent®), a live attenuated influenza vaccine (e.g., FluMist Quadrivalent®), an unadjuvanted influenza vaccine, an adjuvant influenza vaccine, or a subunit or split vaccine.

In some embodiments, a composition comprises a first vaccine that comprises a SARS-CoV-2 vaccine and a second vaccine that comprises an influenza vaccine.

In some embodiments, a composition comprises a first vaccine that comprises BNT162b2 and a second vaccine that comprises Fluzone®, Fluzone high-dose quadrivalent®, Fluzone quadrivalent®, Fluzone intradermal quadrivalent®, or Fluzone quadrivalent southern hemisphere®.

In some embodiments, a composition comprises an RSV vaccine. In some embodiments, a composition comprises a second vaccine that comprises an RSV vaccine.

In some embodiments, a composition comprises a first vaccine that comprises a SARS-CoV-2 vaccine and a second vaccine that comprises an RSV vaccine.

In some embodiments, a composition described herein further comprises a third vaccine.

In some embodiments, a composition comprises a first vaccine, a second vaccine, and a third vaccine, each of which deliver one or more antigens associated with a different virus (e.g., wherein each of the first vaccine, the second vaccine, and the third vaccine deliver an antigen associated with a different respiratory virus).

In some embodiments, a composition comprises a first vaccine that comprises a SARS-CoV-2 vaccine, a second vaccine that comprises an influenza vaccine, and a third vaccine that is or comprises an RSV vaccine.

In some embodiments, a composition comprises a first vaccine, a second vaccine, and optionally a third vaccine, wherein each of the first vaccine, second vaccine, and optional third vaccine are present in an amount that has been shown to provide a clinical benefit to subjects when administered alone (e.g., an amount shown in clinical trials to provide a clinical benefit).

In some embodiments, a composition comprises each of a first vaccine, a second vaccine, and if present, a third vaccine, in an amount of about 100 μl to about 1 mL.

In some embodiments, the combined amount of a first vaccine, a second vaccine, and, if present, a third vaccine in a composition, is not more than 1 mL.

In some embodiments, the combined amount of a first vaccine and a second vaccine in a composition is about 450 μL, about 550 μL, about 700 μL, about 750 μL, or about 800 μL.

In some embodiments, a composition is a pre-filled syringe.

In some embodiments, the present disclosure provides a pre-filled syringe comprising a SARS-CoV-2 vaccine (e.g., BNT162b2).

In some embodiments, provided herein, is a method comprising administering to a subject (i) a composition of described herein, or (ii) the contents of a pre-filled syringe described herein.

In some embodiments, a method described herein is performed in a pharmacy.

In some embodiments, a method described herein is performed in a hospital.

In some embodiments, a composition described herein, and/or the contents of a syringe described herein, is administered via intramuscular (IM) injection.

In some embodiments, a composition produced by a method described herein is administered shortly after being produced (e.g., within an hour or less, 30 minutes or less, 15 minutes or less, 10 minutes or less, 5 minutes or less, or immediately before administering to a subject).

In some embodiments, a method described herein can result in the induction of an immune response in a subject.

In some embodiments, a method described herein can be used as a method of vaccinating a subject.

In some embodiments, described herein, is a comprising (a) a first vaccine, (b) a second vaccine, and (c) instructions for performing a method described herein.

In some embodiments, a kit comprises a first vaccine and a second vaccine, wherein the first vaccine and/or the second vaccine are each provided in a multi-dose vial.

In some embodiments, a kit comprises a first vaccine and a second vaccine, wherein the first vaccine and/or the second vaccine are each provided as a pre-filled syringe.

In some embodiments, a kit further comprises a third vaccine.

In some embodiments, a kit comprises a third vaccine, wherein the third vaccine is provided in a multi-dose vial.

In some embodiments, a kit comprises a third vaccine, wherein the third vaccine is provided in a prefilled syringe.

In some embodiments, a kit comprises a sterile single-packaged adapter (e.g., a Luer-Luer adapter (e.g., a Luer dual female adapter)).

In some embodiments, a method described herein produces a combination vaccine that is sterile.

In some embodiments, a composition described herein is sterile.

In some embodiments, the present disclosure provides a method of preparing and/or administering commingled vaccines, the method comprising:
providing a first vial containing a first vaccine and a second vial containing a second vaccine;
providing an empty syringe comprising a detachable needle assembly;
withdrawing a dosage amount of the first vaccine from the first vial using the syringe;
aseptically removing the needle assembly from the syringe;
providing an unused needle assembly;
aseptically attaching the unused needle assembly to the syringe; and
slowly withdrawing a dosage amount of the second vaccine from the second vial using the syringe, thereby creating a syringe with commingled vaccines.

In some embodiments, a method described herein further comprises administering commingled vaccines to a patient.

In some embodiments, a method described herein comprises slowly withdrawing a dosage amount of a second vaccine from a second vial, wherein the slow withdraw is performed over the course of at least 5 seconds.

In some embodiments, a method described herein uses a first vaccine and a second vaccine, wherein the first vaccine comprises a first delivery modality or payload and the second vaccine comprises a second delivery modality or payload.

### BRIEF DESCRIPTION OF THE DRAWING

FIG. 1 depicts a schematic/workflow for mixing vaccines, according to aspects of the present disclosure.

FIG. 2 illustrates data illustrating minimal loss in vaccine volume, according to aspects of the present disclosure.

FIG. 3 depicts a method for mixing vaccines, according to aspects of the present disclosure.

### CERTAIN DEFINITIONS

About or Approximately: The term “about” or “approximately”, when used herein in reference to a value, refers to a value that is similar, in context to a stated reference value. In general, those skilled in the art, familiar with the context, will appreciate the relevant degree of variance encompassed by “about” or “approximately” in that context. For example, in some embodiments, the term “about” or “approximately” may encompass a range of values that are within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Administration: As used herein, the term “administration” typically refers to the administration of a composition to a subject or system. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, Intradermal, Intradermal, transdermal, etc.), enteral, intra-arterial, intradermal, intragastric, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (e.g., by intratracheal instillation), vaginal, vitreal, etc. In some embodiments, administration may be intramuscular. In some embodiments, administration may involve dosing that Is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Agent. In general, the term “agent”, as used herein, is used to refer to an entity (e.g., a lipid, metal, nucleic acid, polypeptide, polysaccharide, small molecule, etc., or complex, combination, mixture or system [e.g., cell, tissue, organism] thereof), or phenomenon (e.g., heat, electric current or field, magnetic force or field, etc.). In appropriate circumstances, as will be clear from context to those skilled in the art, the term may be utilized to refer to an entity that is or comprises a cell or organism, or a fraction, extract, or component thereof. Alternatively, or additionally, as context will make clear, the term may be used to refer to a natural product in that it is found in and/or is obtained from nature. In some instances, again as will be clear from context, the term may be used to refer to one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents may be provided as collections or libraries, for example that may be screened to identify or characterize active agents within them. In some cases, the term “agent” may refer to a compound or entity that is or comprises a polymer; in some cases, the term may refer to a compound or entity that comprises one or more polymeric moieties. In some embodiments, the term “agent” may refer to a compound or entity that is not a polymer and/or is substantially free of any polymer and/or of one or more particular polymeric moieties. In some embodiments, the term may refer to a compound or entity that lacks or is substantially free of any polymeric moiety.

Analog: As used herein, the term “analog” refers to a substance that shares one or more particular structural features, elements, components, or moleties with a reference substance. Typically, an “analog” shows significant structural similarity with the reference substance, for example sharing a core or consensus structure, but also differs in certain discrete ways. In some embodiments, an analog is a substance that can be generated from the reference substance, e.g., by chemical manipulation of the reference substance. In some embodiments, an analog is a substance that can be generated through performance of a synthetic process substantially similar to (e.g., sharing a plurality of steps with) one that generates the reference substance. In some embodiments, an analog is or can be generated through performance of a synthetic process different from that used to generate the reference substance.

Antibody agent: As used herein, the term “antibody agent” refers to an agent that specifically binds to a particular antigen. In some embodiments, the term encompasses any polypeptide or polypeptide complex that includes immunoglobulin structural elements sufficient to confer specific binding. Exemplary antibody agents include, but are not limited to monoclonal antibodies or polyclonal antibodies. In some embodiments, an antibody agent may include one or more constant region sequences that are characteristic of mouse, rabbit, primate, or human antibodies. In some embodiments, an antibody agent may include one or more sequence elements are humanized, primatized, chimeric, etc., as is known in the art. In many embodiments, the term “antibody agent” is used to refer to one or more of the art-known or developed constructs or formats for utilizing antibody structural and functional features in alternative presentation. For example, embodiments, an antibody agent utilized in accordance with the present disclosure is in a format selected from, but not limited to, intact IgA, IgG, IgE or IgM antibodies; bi- or multi-specific antibodies (e.g., Zybodies®, etc.); antibody fragments such as Fab fragments, Fab′ fragments, F(ab′)2 fragments, Fd′ fragments, Fd fragments, and isolated complementarity determining regions (CDRs) or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPsTM”); single chain or Tandem diabodies (TandAb®); VHHs; Anticalins®; Nanobodies® minibodies; BITE®s; ankyrin repeat proteins or DARPINS®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc.], or other pendant group [e.g., poly-ethylene glycol, etc.]. In many embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments, an included CDR is substantially identical to a reference CDR in that it shows at least 95%, 96%, 97%, 98%, 99%, or 100% sequence identity with the reference CDR. In some embodiments an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an included CDR is substantially identical to a reference CDR in that at least one amino acid within the Included CDR is substituted as compared with the reference CDR but the included CDR has an amino acid sequence that is otherwise identical with that of the reference CDR. In some embodiments, an included CDR Is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain.

Antibody agents can be made by the skilled person using methods and commercially available services and kits known in the art. For example, methods of preparation of monoclonal antibodies are well known in the art and include hybridoma technology and phage display technology. Further antibodies suitable for use in the present disclosure are described, for example, in the following publications: Antibodies A Laboratory Manual, Second edition. Edward A. Greenfield. Cold Spring Harbor Laboratory Press (Sep. 30, 2013); Making and Using Antibodies: A Practical Handbook, Second Edition. Eds. Gary C. Howard and Matthew R. Kaser. CRC Press (Jul. 29, 2013); Antibody Engineering: Methods and Protocols, Second Edition (Methods in Molecular Biology). Patrick Chames. Humana Press (Aug. 21, 2012); Monoclonal Antibodies: Methods and Protocols (Methods in Molecular Biology). Eds. Vincent Ossipow and Nicolas Fischer. Humana Press (Feb. 12, 2014); and Human Monoclonal Antibodies: Methods and Protocols (Methods in Molecular Biology). Michael Steinitz. Humana Press (Sep. 30, 2013).

Antibodies may be produced by standard techniques, for example by immunization with the appropriate polypeptide or portion(s) thereof, or by using a phage display library. If polyclonal antibodies are desired, a selected mammal (e.g., mouse, rabbit, goat, horse, etc.) is immunized with an immunogenic polypeptide bearing a desired epitope(s), optionally haptenized to another polypeptide. Depending on the host species, various adjuvants may be used to increase immunological response. Such adjuvants include, but are not limited to, Freund's, mineral gels such as aluminum hydroxide, and surface-active substances such as lysolecithin, pluronic polyols, polyanions, peptides, oil emulsions, keyhole limpet hemocyanin, and dinitrophenol. Serum from the immunized animal is collected and treated according to known procedures. If serum containing polyclonal antibodies to the desired epitope contains antibodies to other antigens, the polyclonal antibodies can be purified by immunoaffinity chromatography or any other method known in the art. Techniques for producing and processing polyclonal antisera are well known in the art.

Antigen: The term “antigen”, as used herein, refers to (i) an agent that elicits an immune response; and/or (ii) an agent that binds to a T cell receptor (e.g., when presented by an MHC molecule) or to an antibody. In some embodiments, an antigen elicits a humoral response (e.g., including production of antigen-specific antibodies); in some embodiments, an antigen elicits a cellular response (e.g., involving T-cells whose receptors specifically interact with the antigen). In some embodiments, an antigen binds to an antibody and may or may not induce a particular physiological response in an organism. In general, an antigen may be or include any chemical entity such as, for example, a small molecule, a nucleic acid, a polypeptide, a carbohydrate, a lipid, a polymer (in some embodiments other than a biologic polymer (e.g., other than a nucleic acid or amino acid polymer)) etc. In some embodiments, an antigen is or comprises a polypeptide. In some embodiments, an antigen is or comprises a glycan. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source). In some embodiments, antigens utilized in accordance with the present invention are provided in a crude form. In some embodiments, an antigen is a recombinant antigen.

Binding. It will be understood that the term “binding”, as used herein, typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts —including where interacting entities or moieties are studied in isolation or in the context of more complex systems (e.g., while covalently or otherwise associated with a carrier entity and/or in a biological system or cell).

Cap: As used herein, the term “cap” refers to a structure comprising or essentially consisting of a nucleoside-5′-triphosphate that is typically joined to a 5′-end of an uncapped RNA (e.g., an uncapped RNA having a 5′-diphosphate). In some embodiments, a cap is or comprises a guanine nucleotide. In some embodiments, a cap Is or comprises a naturally occurring RNA 5′ cap, Including, e.g., but not limited to a N7-methylguanosine cap, which has a structure designated as “m7G.” In some embodiments, a cap is or comprises a synthetic cap analog that resembles an RNA cap structure and possesses the ability to stabilize RNA if attached thereto, including, e.g., but not limited to anti-reverse cap analogs (ARCAs) known in the art. Those skilled in the art will appreciate that methods for joining a cap to a 5′ end of an RNA are known in the art. For example, in some embodiments, a capped RNA may be obtained by in vitro capping of RNA that has a 5′ triphosphate group or RNA that has a 5′ diphosphate group with a capping enzyme system (including, e.g., but not limited to vaccinia capping enzyme system or Saccharomyces cerevisiae capping enzyme system). Alternatively, a capped RNA can be obtained by in vitro transcription (IVT) of a DNA template, wherein, in addition to the GTP, an IVT system also contains a cap analog, e.g., as known in the art. Non-limiting examples of a cap analog include a m7GpppG cap analog or an N7-methyl-, 2′-O-methyl-GpppG ARCA cap analog or an N7-methyl-, 3′-O-methyl-GpppG ARCA cap analog, or any commercially available cap analogs, including, e.g., CleanCap (Trilink), EZ Cap, etc. In some embodiments, a cap analog is or comprises a trinucleotide cap analog.

Comparable: As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or Indicative of the variation in those features that are varied.

Complementary: As used herein, the term “complementary” is used in reference to oligonucleotide hybridization related by base-pairing rules. For example, the sequence “C-A-G-T” Is complementary to the sequence “G-T-C-A.” Complementarity can be partial or total. Thus, any degree of partial complementarity is intended to be included within the scope of the term “complementary” provided that the partial complementarity permits oligonucleotide hybridization. Partial complementarity is where one or more nucleic acid bases is not matched according to the base pairing rules. Total or complete complementarity between nucleic acids is where each and every nucleic acid base is matched with another base under the base pairing rules.

Detecting: The term “detecting” is used broadly herein to Include appropriate means of determining the presence or absence of an entity of interest or any form of measurement of an entity of interest in a sample. Thus, “detecting” may include determining, measuring, assessing, or assaying the presence or absence, level, amount, and/or location of an entity of interest. Quantitative and qualitative determinations, measurements or assessments are included, including semi-quantitative. Such determinations, measurements or assessments may be relative, for example when an entity of interest is being detected relative to a control reference, or absolute. As such, the term “quantifying” when used in the context of quantifying an entity of interest can refer to absolute or to relative quantification. Absolute quantification may be accomplished by correlating a detected level of an entity of Interest to known control standards (e.g., through generation of a standard curve). Alternatively, relative quantification can be accomplished by comparison of detected levels or amounts between two or more different entities of interest to provide a relative quantification of each of the two or more different entities of interest, i.e., relative to each other.

Determine: Those of ordinary skill in the art, reading the present specification, will appreciate that a step of “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

Dosage form or unit dosage form: Those skilled in the art will appreciate that the term “dosage form” may be used to refer to a physically discrete unit of an active agent (e.g., a therapeutic or diagnostic agent) for administration to a subject. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular subject is determined by one or more attending physicians and may involve administration of multiple dosage forms.

Encapsulate: The term “encapsulate” or “encapsulation” is used herein to refer to at least a portion of a component is enclosed or surrounded by another material or another component in a composition. In some embodiments, a component can be fully enclosed or surrounded by another material or another component in a composition.

Excipient: As used herein, the term “excipient” refers to a non-therapeutic agent that may be included in a pharmaceutical composition, for example to provide or contribute to a desired property or effect (e.g., desired consistency, delivery, and/or stabilizing effect, etc.). In some embodiments, suitable pharmaceutical excipients to be added to a LNP composition may include, for example, salts, starch, glucose, lactose, sucrose, gelatin, sodium chloride, glycerol, propylene, glycol, water, ethanol and the like.

Encode: As used herein, the term “encode” or “encoding” refers to sequence information of a first molecule that guides production of a second molecule having a defined sequence of nucleotides (e.g., mRNA) or a defined sequence of amino acids. For example, a DNA molecule can encode an RNA molecule (e.g., by a transcription process that includes a DNA-dependent RNA polymerase enzyme). An RNA molecule can encode a polypeptide (e.g., by a translation process). Thus, a gene, a cDNA, or a single-stranded RNA (e.g., an mRNA) encodes a polypeptide if transcription and translation of mRNA corresponding to that gene produces the polypeptide in a cell or other biological system. In some embodiments, a coding region of a single-stranded RNA encoding a target polypeptide agent refers to a coding strand, the nucleotide sequence of which is identical to the mRNA sequence of such a target polypeptide agent. In some embodiments, a coding region of a single-stranded RNA encoding a target polypeptide agent refers to a non-coding strand of such a target polypeptide agent, which may be used as a template for transcription of a gene or cDNA.

Expression. As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification.

Functional: As used herein, a “functional” biological molecule is a biological molecule in a form in which it exhibits a property and/or activity by which it is characterized. In some embodiments, a biological molecule may have two functions (i.e., bifunctional) or many functions (i.e., multifunctional).

Gene: As used herein, the term “gene” refers to a DNA sequence in a chromosome that codes for a product (e.g., an RNA product and/or a polypeptide product). In some embodiments, a gene includes coding sequence (i.e., sequence that encodes a particular product); in some embodiments, a gene includes non-coding sequence. In some particular embodiments, a gene may include both coding (e.g., exonic) and non-coding (e.g., intronic) sequences. In some embodiments, a gene may include one or more regulatory elements that, for example, may control or impact one or more aspects of gene expression (e.g., cell-type-specific expression, inducible expression, etc.).

Gene product or expression product: As used herein, the term “gene product” or “expression product” generally refers to an RNA transcribed from the gene (pre-and/or post-processing) or a polypeptide (pre-and/or post-modification) encoded by an RNA transcribed from the gene.

Homology: As used herein, the term “homology” or “homolog” refers to the overall relatedness between polynucleotide molecules (e.g., DNA molecules and/or RNA molecules) and/or between polypeptide molecules. In some embodiments, polynucleotide molecules (e.g., DNA molecules and/or RNA molecules) and/or polypeptide molecules are considered to be “homologous” to one another if their sequences are at least 15%, 20%, 25%, 30%, 35%, 40%, 45%, 50%, 55%, 60%, 65%, 70%, 75%, 80%, 85%, 90%, 95%, or 99% identical. In some embodiments, polynucleotide molecules (e.g., DNA molecules and/or RNA molecules) and/or polypeptide molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar (e.g., containing residues with related chemical properties at corresponding positions). For example, as is well known by those of ordinary skill in the art, certain amino acids are typically classified as similar to one another as “hydrophobic” or “hydrophilic” amino acids, and/or as having “polar” or “non-polar” side chains. Substitution of one amino acid for another of the same type may often be considered a “homologous” substitution.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna. CMP matrix.

Improved, increased or reduced: As used herein, these terms, or grammatically comparable comparative terms, indicate values that are relative to a comparable reference measurement. For example, in some embodiments, an assessed value achieved with an agent of interest may be “improved” relative to that obtained with a comparable reference agent. Alternatively or additionally, in some embodiments, an assessed value achieved in a subject or system of interest may be “improved” relative to that obtained in the same subject or system under different conditions (e.g., prior to or after an event such as administration of an agent of interest), or in a different, comparable subject (e.g., in a comparable subject or system that differs from the subject or system of interest in presence of one or more indicators of a particular disease, disorder or condition of interest, or in prior exposure to a condition or agent, etc.). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

In vitro: The term “in vitro” as used herein refers to events that occur in an artificial environment, e.g., in a test tube or reaction vessel (e.g., a bioreactor), in cell culture, etc., rather than within a multi-cellular organism.

In vitro transcription: As used herein, the term “in vitro transcription” or “IVT” refers to the process whereby transcription occurs in vitro in a non-cellular system to produce a synthetic RNA product for use in various applications, including, e.g., production of protein or polypeptides. Such synthetic RNA products can be translated in vitro or introduced directly into cells, where they can be translated. Such synthetic RNA products include, e.g., but not limited to mRNAs, antisense RNA molecules, shRNA molecules, long non-coding RNA molecules, ribozymes, aptamers, guide RNAs (e.g., for CRISPR), ribosomal RNAs, small nuclear RNAs, small nucleolar RNAS, and the like. An IVT reaction typically utilizes a DNA template (e.g., a linear DNA template) as described and/or utilized herein, ribonucleotides (e.g., non-modified ribonucleotide triphosphates or modified ribonucleotide triphosphates), and an appropriate RNA polymerase.

In vitro transcription RNA composition: As used herein, the term “in vitro transcription RNA composition” refers to a composition comprising target RNA synthesized by in vitro transcription. In some embodiments, such a composition can comprise excess in vitro transcription reagents (including, e.g., ribonucleotides and/or capping agents), nucleic acids or fragments thereof such as DNA templates or fragments thereof, polypeptides or fragments thereof such as recombinant enzymes or host cell proteins or fragments thereof, and/or other Impurities. In some embodiments, an in vitro transcription RNA composition may have been treated and/or processed prior to one or more purification processes that ultimately produces an RNA transcript preparation comprising RNA transcript at a desired concentration in an appropriate buffer for formulation and/or further manufacturing and/or processing. For example, in some embodiments, an in vitro transcription RNA composition may have been treated to remove or digest DNA template (e.g., using a DNase). In some embodiments, an in vitro transcription RNA composition may have been treated to remove or digest polypeptides (e.g., enzymes such as RNA polymerases, RNase inhibitors, etc.) present in an in vitro transcription reaction (e.g., using a protease).

In vivo: As used herein, the term “in vivo” refers to events that occur within a multi-cellular organism, such as a human and a non-human animal.

Nanoparticle: As used herein, the term “nanoparticle” refers to a particle having a diameter of less than 1000 nanometers (nm). In some embodiments, a nanoparticle has a diameter of less than 300 nm, as defined by the National Science Foundation. In some embodiments, a nanoparticle has a diameter of less than 100 nm as defined by the National Institutes of Health. In some embodiments, a nanoparticle has a diameter of less than 80 nm as defined by the National Institutes of Health. In some embodiments, a nanoparticle comprises one or more enclosed compartments, separated from the bulk solution by a membrane, which surrounds and encloses a space or compartment.

Nucleic acid/Polynucleotide: As used herein, the term “nucleic acid” refers to a polymer of at least 2 nucleotides or more, including, e.g., at least 3 nucleotides, at least 4 nucleotides, at least 5 nucleotides, at least 6 nucleotides, at least 7 nucleotides, at least 8 nucleotides, at least 9 nucleotides, at least 10 nucleotides, or more. In some embodiments, a nucleic acid is or comprises DNA. In some embodiments, a nucleic acid is or comprises RNA. In some embodiments, a nucleic acid is or comprises peptide nucleic acid (PNA). In some embodiments, a nucleic acid is or comprises a single stranded nucleic acid. In some embodiments, a nucleic acid is or comprises a double-stranded nucleic acid. In some embodiments, a nucleic acid comprises both single and double-stranded portions. In some embodiments, a nucleic acid comprises a backbone that comprises one or more phosphodiester linkages. In some embodiments, a nucleic acid comprises a backbone that comprises both phosphodiester and non-phosphodiester linkages. For example, in some embodiments, a nucleic acid may comprise a backbone that comprises one or more phosphorothioate or 5′-N-phosphoramidite linkages and/or one or more peptide bonds, e.g., as in a “peptide nucleic acid”. In some embodiments, a nucleic acid comprises one or more, or all, natural residues (e.g., adenine, cytosine, deoxyadenosine, deoxycytidine, deoxyguanosine, deoxythymidine, guanine, thymine, uracil). In some embodiments, a nucleic acid comprises on or more, or all, non-natural residues. In some embodiments, a non-natural residue comprises a nucleoside analog (e.g., 2-aminoadenosine, 2-thiothymidine, inosine, pyrrolo-pyrimidine, 3-methyl adenosine, 5-methylcytidine, C-5 propynyl-cytidine, 1-methyl-pseudouridine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 6-O-methylguanine, 2-thiocytidine, methylated bases, intercalated bases, and combinations thereof). In some embodiments, a non-natural residue comprises one or more modified sugars (e.g., 2′-fluororibose, ribose, 2′-deoxyribose, arabinose, and hexose) as compared to those in natural residues. In some embodiments, a nucleic acid has a nucleotide sequence that encodes a functional gene product such as an RNA or polypeptide. In some embodiments, a nucleic acid has a nucleotide sequence that comprises one or more introns.

In some embodiments, a nucleic acid may be prepared by isolation from a natural source, enzymatic synthesis (e.g., by polymerization based on a complementary template, e.g., in vivo or in vitro, reproduction in a recombinant cell or system, or chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 225, 250, 275, 300, 325, 350, 375, 400, 425, 450, 475, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, or 20,000 or more residues or nucleotides long.

Pharmaceutical grade: The term “pharmaceutical grade” as used herein refers to standards for chemical and biological drug substances, drug products, dosage forms, compounded preparations, excipients, medical devices, and dietary supplements, established by a recognized national or regional pharmacopeia (e.g., The United States Pharmacopeia and The Formulary (USP-NF)).

Polypeptide: The term “polypeptide”, as used herein, typically has its art-recognized meaning of a polymer of at least three amino acids or more. Those of ordinary skill in the art will appreciate that the term “polypeptide” is intended to be sufficiently general as to encompass not only polypeptides having a complete sequence recited herein, but also to encompass polypeptides that represent functional, biologically active, or characteristic fragments, portions or domains (e.g., fragments, portions, or domains retaining at least one activity) of such complete polypeptides. In some embodiments, polypeptides may contain L-amino acids, D-amino acids, or both and/or may contain any of a variety of amino acid modifications or analogs known in the art. Useful modifications include, e.g., terminal acetylation, amidation, methylation, etc. In some embodiments, polypeptides may comprise natural amino acids, non-natural amino acids, synthetic amino acids, and combinations thereof (e.g., may be or comprise peptidomimetics). In some embodiments, a polypeptide may be or comprise an enzyme. In some embodiments, a polypeptide may be or comprise a polypeptide antigen. In some embodiments, a polypeptide may be or comprise an antibody agent. In some embodiments a polypeptide may be or comprise a cytokine.

Pure or Purified: As used herein, an agent or entity is “pure” or “purified” if it is substantially free of other components. For example, a preparation that contains more than about 90% of a particular agent or entity is typically considered to be a pure preparation. In some embodiments, an agent or entity is at least 91%, at least 92%, at least 93%, at least 94%, at least 95%, at least 96%, at least 97%, at least 98%, or at least 99% pure in a preparation.

Ribonucleotide: As used herein, the term “ribonucleotide” encompasses unmodified ribonucleotides and modified ribonucleotides. For example, unmodified ribonucleotides include the purine bases adenine (A) and guanine (G), and the pyrimidine bases cytosine (C) and uracil (U). Modified ribonucleotides may include one or more modifications including, but not limited to, for example, (a) end modifications, e.g., 5′ end modifications (e.g., phosphorylation, dephosphorylation, conjugation, inverted linkages, etc.), 3′ end modifications (e.g., conjugation, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, and (d) internucleoside linkage modifications, including modification or replacement of the phosphodiester linkages. The term “ribonucleotide” also encompasses ribonucleotide triphosphates including modified and non-modified ribonucleotide triphosphates.

Ribonucleic acid (RNA): As used herein, the term “RNA” refers to a polymer of ribonucleotides. In some embodiments, an RNA is single stranded. In some embodiments, an RNA is double stranded. In some embodiments, an RNA comprises both single and double stranded portions. In some embodiments, an RNA can comprise a backbone structure as described in the definition of “Nucleic acid/Polynucleotide” above. An RNA can be a regulatory RNA (e.g., siRNA, microRNA, etc.), or a messenger RNA (mRNA). In some embodiments, an RNA is an mRNA. In some embodiments, where an RNA is a mRNA, an RNA typically comprises at its 3′ end a poly(A) region. In some embodiments where an RNA is a mRNA, an RNA typically comprises at its 5′ end, an art-recognized cap structure, e.g., for recognizing and attachment of a mRNA to a ribosome to initiate translation. In some embodiments, an RNA is a synthetic RNA. Synthetic RNAs include RNAs that are synthesized in vitro (e.g., by enzymatic synthesis methods and/or by chemical synthesis methods). In some embodiments, an RNA is a single-stranded RNA. In some embodiments, a single-stranded RNA may comprise self-complementary elements and/or may establish a secondary and/or tertiary structure. One of ordinary skill in the art will understand that when a single-stranded RNA is referred to as “encoding,” it can mean that it comprises a nucleic acid sequence that itself encodes or that it comprises a complement of the nucleic acid sequence that encodes. In some embodiments, a single-stranded RNA can be a self-amplifying RNA (also known as self-replicating RNA).

Recombinant. as used herein, is Intended to refer to polypeptides that are designed, engineered, prepared, expressed, created, manufactured, and/or or Isolated by recombinant means, such as polypeptides expressed using a recombinant expression vector transfected into a host cell; polypeptides isolated from a recombinant, combinatorial human polypeptide library; polypeptides isolated from an animal (e.g., a mouse, rabbit, sheep, fish, etc.) that is transgenic for or otherwise has been manipulated to express a gene or genes, or gene components that encode and/or direct expression of the polypeptide or one or more component(s), portion(s), element(s), or domain(s) thereof; and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of the polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc.).

Reference: As used herein, the term “reference” describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

RNA polymerase: As used herein, the term “RNA polymerase” refers to an enzyme that catalyzes polyribonucleotide synthesis by addition of ribonucleotide units to a nucleotide chain using DNA or RNA as a template. The term refers to either a complete enzyme as it occurs in nature, or an isolated, active catalytic or functional domain, or fragment thereof. In some embodiments, an RNA polymerase enzyme initiates synthesis at the 3′-end of a primer or a nucleic acid strand, or at a promoter sequence, and proceeds in the 5′-direction along the target nucleic acid to synthesize a strand complementary to the target nucleic acid until synthesis terminates.

RNA transcript preparation: The term “RNA transcript preparation” as used herein refers to a preparation comprising RNA transcript that is purified from an in vitro transcription RNA composition described herein. In some embodiments, an RNA transcript preparation is a preparation comprising pharmaceutical-grade RNA transcript. In some embodiments, an RNA transcript preparation is a preparation comprising RNA transcript, in which one or more product quality attributes are characterized and determined to meet a release and/or acceptance criteria (e.g., as described herein). Examples of such product quality attributes include, but are not limited to appearance, RNA length, Identity of drug substance as RNA, RNA integrity, RNA sequence, RNA concentration, pH, osmolality, residual DNA template, residual double stranded RNA, bacterial endotoxins, bioburden, and combinations thereof.

Room temperature: As used herein, the term “room temperature” refers to an ambient temperature. In some embodiments, a room temperature is about 18 °C-30 °C, e.g., about 18 °C-25 °C, or about 20 °C-25 °C, or about 20-30 °C, or about 23-27 °C or about 25 °C.

Sample: As used herein, the term “sample” typically refers to an aliquot of material obtained or derived from a source of interest, e.g., as described herein. In some embodiments, a source of interest is a biological or environmental source. In some embodiments, a source of interest may be or comprise a cell or an organism, such as a microbe, a plant, or an animal (e.g., a mouse). In some embodiments, a source of interest is or comprises biological tissue or fluid. In some embodiments, a biological fluid may be or comprise an intracellular fluid, an extracellular fluid, an intravascular fluid (blood plasma), an interstitial fluid, a lymphatic fluid, and/or a transcellular fluid. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., bronchoalveolar, ductal, nasal, ocular, oral, uterine, vaginal, or other washing or lavage). In some embodiments, a sample is or comprises cells obtained from a subject. In some embodiments, a sample is a “primary sample” obtained directly from a source of interest by any appropriate means. In some embodiments, as will be clear from context, the term “sample” refers to a preparation that Is obtained by processing (e.g., by removing one or more components of and/or by adding one or more agents to) a primary sample. For example, a “processed sample” may comprise, for example nucleic acids or proteins extracted from a sample or obtained by subjecting a primary sample to one or more techniques such as amplification or reverse transcription of nucleic acid, isolation and/or purification of certain components, etc.

Stable: The term “stable,” when applied to nucleic acids and/or compositions comprising nucleic acids, e.g., encapsulated in lipid nanoparticles, means that such nucleic acids and/or compositions maintain one or more aspects of their characteristics (e.g., physical and/or structural characteristics, function, and/or activity) over a period of time under a designated set of conditions (e.g., pH, temperature, light, relative humidity, etc.). In some embodiments, such stability is maintained over a period of time of at least about one hour; in some embodiments, such stability is maintained over a period of time of about 5 hours, about 10 hours, about one (1) day, about one (1) week, about two (2) weeks, about one (1) month, about two (2) months, about three (3) months, about four (4) months, about five (5) months, about six (6) months, about eight (8) months, about ten (10) months, about twelve (12) months, about twenty-four (24) months, about thirty-six (36) months, or longer. In some embodiments, such stability is maintained over a period of time within the range of about one (1) day to about twenty-four (24) months, about two (2) weeks to about twelve (12) months, about two (2) months to about five (5) months, etc. In some embodiments, such stability is maintained under an ambient condition (e.g., at room temperature and ambient pressure). In some embodiments, such stability is maintained under a physiological condition (e.g., in vivo or at about 37 °C for example in serum or in phosphate buffered saline). In some embodiments, such stability is maintained under cold storage (e.g., at or below about 4 °C, including, e.g., −20 °C, or −70 °C). In some embodiments, such stability is maintained when nucleic acids and/or compositions comprising the same are protected from light (e.g., maintaining in the dark).

As an example, in some embodiments, the term “stable” is used in reference to a nanopartide composition (e.g., a lipid nanoparticle composition). In such embodiments, a stable nanoparticle composition (e.g., a stable nanoparticle composition) and/or component(s) thereof maintain one or more aspects of its characteristics (e.g., physical and/or structural characteristics, function(s), and/or activity) over a period of time under a designated set of conditions. For example, in some embodiments, a stable nanoparticle composition (e.g., a lipid nanoparticle composition) is characterized in that average particle size, particle size distribution, and/or polydispersity of nanoparticles is substantially maintained (e.g., within 10% or less, as compared to the initial characteristic(s)) over a period of time (e.g., as described herein) under a designated set of conditions (e.g., as described herein). In some embodiments, a stable nanoparticle composition (e.g., a lipid nanoparticle composition) is characterized in that no detectable amount of degradation products (e.g., associated with hydrolysis and/or enzymatic digestion) is present after it is maintained under a designated set of conditions (e.g., as described herein) over a period of time.

Synthetic: As used herein, the term “synthetic” refers to an entity that is artificial, or that is made with human intervention, or that results from synthesis rather than naturally occurring. For example, in some embodiments, a synthetic nucleic acid or polynucleotide refers to a nucleic acid molecule that is chemically synthesized, e.g., in some embodiments by solid-phase synthesis. In some embodiments, the term “synthetic” refers to an entity that is made outside of biological cells. For example, in some embodiments, a synthetic nucleic acid or polynucleotide refers to a nucleic acid molecule (e.g., an RNA) that is produced by in vitro transcription using a template.

Threshold level (e.g., acceptance criteria): as used herein, the term “threshold level” refers to a level that are used as a reference to attain information on and/or classify the results of a measurement, for example, the results of a measurement attained in an assay. For example, in some embodiments, a threshold level means a value measured in an assay that defines the dividing line between two subsets of a population (e.g., a batch that satisfy quality control criteria vs. a batch that does not satisfy quality control criteria). Thus, a value that is equal to or higher than the threshold level defines one subset of the population, and a value that is lower than the threshold level defines the other subset of the population. A threshold level can be determined based on one or more control samples or across a population of control samples. A threshold level can be determined prior to, concurrently with, or after the measurement of interest is taken. In some embodiments, a threshold level can be a range of values.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Green and Sambrook, Molecular Cloning: A Laboratory Manual (4th ed., Cold Spring Harbor Laboratory Press, Cold Spring Harbor, N.Y. (2012)), which is incorporated herein by reference for any purpose.

### DETAILED DESCRIPTION OF CERTAIN EMBODIMENTS

Recent developments in vaccine technologies have led to an increase in the number of vaccines that are available to patients. These vaccines often include various mechanisms and/or delivery modalities for reaching their targets. For example, nucleic acid therapeutics, and particularly RNA therapeutics represent a particularly promising class of therapies for treatment and prevention of various diseases such as cancer, infectious diseases, and/or diseases or disorders associated with overabundance or deficiency in certain proteins.

RNA therapeutics in particular provide remarkably effective as vaccines to address the COVID19 pandemic. Particularly given the promise of this technology, and its adaptability to a wide variety of clinical contexts, including massively large scale (e.g., vaccination and/or treatment on a global scale such as is under development for SARS-CoV-2), improvements to manufacturing technologies, especially those applicable to large-scale production, are especially valuable.

Development of effective delivery technologies has been central to the success of nucleic acid therapeutics, and lipid nanoparticle technologies have proven to be particularly effective (reviewed in, for example, Cullis et al. Molecular Therapy 25:1467, Jul. 5, 2017; See also, U.S. Pat. No. 8,058,069), specifically Including for RNA therapeutics (reviewed in, for example, Hou et al., Nat. Rev. Mater doi.org/10.1038/s41578-021-00358-0, Aug. 10, 2021).

Technologies provided herein are useful, among other things, to achieve particularly effective and/or efficient production, e.g., on commercial scale and/or under commercial conditions, of pharmaceutical grade LNP preparations and/or compositions (e.g., nucleic acid-LNP preparations, and specifically RNA-LNP preparations). For example, in various embodiments, provided technologies permit and/or facilitate achievement of requirements unique to pharmaceutical-grade (and/or scale) production such as, for example, batch size and/or rate of production, pre-determined in-process controls and/or lot release specifications (e.g., high purity, Integrity, potency, and/or stability, etc.), etc.

The present disclosure provides technologies for manufacturing LNP compositions (e.g., including RNA, e.g., therapeutic RNA such as therapeutic mRNA). In some embodiments, provided technologies are useful for manufacturing pharmaceutical-grade RNA-LNP therapeutics.

In some embodiments, provided technologies are useful for large scale manufacturing of LNP (e.g., nucleic acid-LNP, e.g., RNA-LNP) therapeutics, e.g., pharmaceutical-grade therapeutics. For example, in some such embodiments, technologies provided herein can be used to produce a pharmaceutical-grade batch throughput of at least 10,000 vials of LNP(e.g., nucleic acid-LNP, e.g., RNA-LNP) therapeutics (including, e.g., at least 20,000 vials, at least 30,000 vials, at least 40,000 vials, at least 50,000 vials, at least 60,000 vials, at least 70,000 vials, at least 80,000 vials, at least 90,000 vials, at least 100,000 vials, at least 200,000 vials, at least 300,000 vials, at least 400,000 vials, at least 500,000 vials, or more). For example, in some such embodiments, technologies provided herein can be used to produce a pharmaceutical-grade batch throughput of at least 50 L of LNP (e.g., nucleic acid-LNP, e.g., RNA-LNP) therapeutics (including e.g., at least 50 L, at least 60 L, at least 70 L, at least 80 L, at least 100 L, at least 110 L, at least 120 L, at least 130 L, at least 140 L, at least 150 L or more. In some embodiments, each vial can comprise an RNA drug product in an amount of 0.01 mg to 0.5 mg (e.g., 0.01 mg, 0.02 mg, 0.03 mg, 0.04 mg, 0.05 mg, 0.06 mg, 0.07 mg, 0.08 mg, 0.09 mg, 0.1 mg, 0.15 mg, 0.2 mg, 0.25 mg, 0.3 mg, 0.35 mg, 0.4 mg, 0.45 mg, 0.5 mg).

Technologies described herein can be useful for manufacturing LNP (e.g., nucleic acid-LNP, e.g., RNA-LNP) compositions for treatment and/or prevention of a disease, disorder, or condition (e.g., cancer, infectious diseases, diseases associated with protein deficiency, etc.). In some embodiments, technologies described herein can be useful for manufacturing LNP (e.g., nucleic acid-LNP, e.g., RNA-LNP) compositions that comprise or deliver (e.g., by comprising and/or delivering a nucleic acid, such as an RNA, that encodes it) a polypeptide.

In some particular embodiments, technologies described herein can be useful for manufacturing LNP (e.g., nucleic acid-LNP, e.g., RNA-LNP) compositions for inducing an immune response to an antigen. In some embodiments, technologies described herein can be useful for manufacturing LNP (e.g., nucleic acid-LNP, e.g., RNA-LNP) compositions for treatment and/or prevention of coronavirus infection, e.g., SARS-CoV-2 infection, as described in Walsh et al. “RNA-based COVID-19 vaccine BNT162b2 selected for a pivotal efficacy study” medRxiv preprint (2020), which is online accessible at: https://doi.org/10.1101/2020.08.17.20176651; and Milligan et al. “Phase I/II study of COVID-19 RNA vaccine BNT162b1 in adults” Nature (2020 August), which is online accessible at: https://doi.org/10.1038/s41586-020-2639-4, the contents of each of which are incorporated by reference in their entirety.

### Lipid Nanoparticles

Those skilled in the art are aware that lipid nanoparticles have achieved successful clinical delivery of a wide range of therapeutic agents including, for example, small molecules, and various nucleic acids—e.g., oligonucleotides, siRNAs, and mRNAs (reviewed, for example, in Hu et al., Nat. Rev. Mater. https://doi.org/10.1038/s41578-021-00358-0, Aug. 10, 2021).

Various routes of administration for lipid nanoparticle compositions have been proposed and/or tested; those skilled in the art will be aware of appropriate routes for particular compositions (e.g., depending on agent being delivered). To give but a few examples, in some embodiments, LNPs are parenterally administered; most clinical studies have utilized parenteral administration, and particularly intravenous, subcutaneous, intradermal, intravitreal, intratumoral, or intramuscular injection. Intrautero injection has also been described. In some embodiments, topical administration is utilized. In some embodiments, intranasal administration is utilized.

In some embodiments, administered LNPs are delivered to or accumulate in the liver. Given that the liver is naturally effective at producing and secreting proteins, liver delivery can prove useful for achieving delivery of an LNP-encapsulated agent (and/or, in the case of a nucleic acid agent such as an RNA agent, a polypeptide encoded thereby) into the bloodstream. Such liver delivery has been proposed to be particularly useful, for example, for expression of proteins that are missing in certain metabolic or hematological disorders, or that are effective in provoking immune responses (e.g., particularly antibody responses), for example against infectious agents or cancer cells.

In some embodiments, administered LNPs are delivered to and/or taken up by antigen-presenting cells (e.g., as may be present in skin, muscle, mucosal tissues, etc.); such administration may be particularly useful or effective for induction of T cell immunity (e.g., for treatment of infectious diseases and/or cancers).

In various embodiments, lipid nanoparticles can have an average size (e.g., mean diameter) of about 30 nm to about 150 nm, about 40 nm to about 150 nm, about 50 nm to about 150 nm, about 50 nm to about 130 nm, about 50 nm to about 110 nm, about 50 nm to about 100 nm, about 50 to about 90 nm, or about 60 nm to about 80 nm, or about 60 nm to about 70 nm. In some embodiments, lipid nanoparticles that may be useful in accordance with the present disclosure can have an average size (e.g., mean diameter) of about 50 nm to about 100 nm. In some embodiments, lipid nanoparticles may have an average size (e.g., mean diameter) of less than 80 nm, less than 75 nm, less than 70 nm, less than 65 nm, less than 60 nm, less than 55 nm, less than 50 nm, or less than 45 nm. In some embodiments, lipid nanoparticles that may be useful in accordance with the present disclosure can have an average size (e.g., mean diameter) of about 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm.

In some embodiments, lipids that form lipid nanoparticles described herein comprise: a polymer-conjugated lipid; a cationic lipid; and a helper neutral lipid. In some such embodiments, total polymer-conjugated lipid may be present in about 0.5-5 mol %, about 0.7-3.5 mol %, about 1-2.5 mol %, about 1.5-2 mol %, or about 1.5-1.8 mol % of the total lipids. In some embodiments, total polymer-conjugated lipid may be present in about 1-2.5 mol % of the total lipids. In some embodiments, the molar ratio of total cationic lipid to total polymer-conjugated lipid (e.g., PEG-conjugated lipid) may be about 100:1 to about 20:1, or about 50:1 to about 20:1, or about 40:1 to about 20:1, or about 35:1 to about 25:1. In some embodiments, the molar ratio of total cationic lipid to total polymer-conjugated lipid may be about 35:1 to about 25:1.

In some embodiments involving a polymer-conjugated lipid, a cationic lipid, and a helper neutral lipid in lipid nanoparticles described herein, total cationic lipid is present in about 35-65 mol %, about 40-60 mol %, about 41-49 mol %, about 41-48 mol %, about 42-48 mol %, about 43-48 mol %, about 44-48 mol %, about 45-48 mol %, or about 46-49 mol % of the total lipids. In certain embodiments, total cationic lipid is present in about 47.0, 47.1, 47.2, 47.3, 47.4, 47.5, 47.6, 47.7, 47.8, 47.9 or 48.0 mol % of the total lipids.

In some embodiments involving a polymer-conjugated lipid, a cationic lipid, and a helper neutral lipid in lipid nanoparticles described herein, total neutral lipid is present in about 35-65 mol %, about 40-60 mol %, about 45-55 mol %, or about 47-52 mol % of the total lipids. In some embodiments, total neutral lipid is present in 35-65 mol % of the total lipids. In some embodiments, total non-steroid neutral lipid (e.g., DPSC) is present in about 5-15 mol %, about 7-13 mol %, or 9-11 mol % of the total lipids. In some embodiments, total non-steroid neutral lipid Is present in about 9.5, 10 or 10.5 mol % of the total lipids. In some embodiments, the molar ratio of the total cationic lipid to the non-steroid neutral lipid ranges from about 4.1:1.0 to about 4.9:1.0, from about 4.5:1.0 to about 4.8:1.0, or from about 4.7:1.0 to 4.8:1.0. In some embodiments, total steroid neutral lipid (e.g., cholesterol) is present in about 35-50 mol %, about 39-49 mol %, about 39-46 mol %, about 39-44 mol %, or about 39-42 mol % of the total lipids. In certain embodiments, total steroid neutral lipid (e.g., cholesterol) is present in about 39, 40, 41, 42, 43, 44, 45, or 46 mol % of the total lipids. In certain embodiments, the molar ratio of total cationic lipid to total steroid neutral lipid is about 1.5:1 to 1:1.2, or about 1.2:1 to 1:1.2.

In some embodiments, a lipid composition comprising a cationic lipid, a polymer-conjugated lipid, and a neutral lipid can have individual lipids present in certain molar percents of the total lipids, or in certain molar ratios (relative to each other).

In some embodiments, lipids that form the lipid nanoparticles comprise: a polymer-conjugated lipid (e.g., PEG-conjugated lipid); a cationic lipid; and a neutral lipid, wherein the polymer-conjugated lipid is present in about 1-2.5 mol % of the total lipids; the cationic lipid is present in 35-65 mol % of the total lipids; and the neutral lipid is present in 35-65 mol % of the total lipids. In some embodiments, lipids that form the lipid nanoparticles comprise: a polymer-conjugated lipid (e.g., PEG-conjugated lipid); a cationic lipid; and a neutral lipid, wherein the polymer-conjugated lipid is present in about 1-2 mol % of the total lipids; the cationic lipid is present in 45-48.5 mol % of the total lipids; and the neutral lipid is present in 45-55 mol % of the total lipids. In some embodiments, lipids that form the lipid nanoparticles comprise: a polymer-conjugated lipid (e.g., PEG-conjugated lipid); a cationic lipid; and a neutral lipid comprising a non-steroid neutral lipid and a steroid neutral lipid, wherein the polymer-conjugated lipid is present in about 1-2 mol % of the total lipids; the cationic lipid is present in 45-48.5 mol % of the total lipids; the non-steroid neutral lipid is present in 9-11 mol % of the total lipids; and the steroid neutral lipid is present in about 36-44 mol % of the total lipids. In many of such embodiments, a neutral lipid comprises DSPC and cholesterol, wherein DSPC is a non-steroid neutral lipid and cholesterol is a steroid neutral lipid.

In some embodiments, lipid nanoparticles include one or more cationic lipids (e.g., ones described herein). In some embodiments, cationic lipid nanoparticles may comprise at least one cationic lipid, at least one polymer-conjugated lipid, and at least one helper lipid (e.g., at least one neutral lipid).

### Liposomes

Those skilled in the art are aware that liposomes have also achieved successful clinical delivery of a wide range of therapeutic agents. In some embodiments, liposomes may include both a hydrophilic region and a hydrophobic region. In some embodiments, liposomes may include a spherical lipid bilayer. In some embodiments, liposomes may include phospholipids (e.g., phosphatidylcholine and/or cholesterol). In some embodiments, liposomes may include phosphatidylethanolamine. Liposomes can be used in connection with drug delivery.

### Lipoplexes

Those skilled in the art are aware that lipoplexes have also achieved successful clinical delivery of a wide range of therapeutic agents. In some embodiments, lipoplexes include cationic lipids. In some aspects according to the present disclosure, cationic lipids are structured to allow or enable electrostatic interaction negatively charges (e.g., negatively charged phosphate backbones (e.g., of nucleic acids)). Accordingly, lipoplexes may be used in connection with the delivery of nucleic acids (i.e., for therapeutic benefit and/or in connection with drug/vaccine delivery).

In the context of the present disclosure, the terms “lipoplex” and “RNA lipoplex particle” relates to a particle that contains lipid, in particular cationic lipid, and RNA. Electrostatic interactions between positively charged liposomes and negatively charged RNA results in complexation and spontaneous formation of RNA lipoplex particles. Positively charged liposomes may be generally synthesized using a cationic lipid, such as DOTMA (dioleoyl-3-trimethylammonium propane), and additional lipids, such as DOPE (dioleoylphosphatidylethanolamine). In some embodiments, a lipid preparation or solution includes 0.6 mM DOTMA, DOPE, ethanol, and acetic acid. In one embodiment, an RNA lipoplex particle is a nanoparticle. The disclosures of U.S. Pat. Nos. 11,173,120 and 11,395,799 are incorporated herein by reference, in their entireties.

### Oligosaccharides

Those skilled in the art are aware that cationic and/or ionizable oligosaccharide complexes have also proven useful for targeted delivery of biological agents, including small molecules, nucleic acids, and/or RNA. According to aspects of the present disclosure, a vaccine as described herein may also include one or more oligosaccharide complexes and/or compounds for delivering a biological agent. In some embodiments, oligosaccharide complexes and/or compounds may include both a cationic oligosaccharide and an RNA component. In some embodiments, oligosaccharide complexes and/or compounds may include an immunomodulator. In some embodiments, oligosaccharide complexes and/or compounds may include disulfide oligosaccharide complexes and/or compounds.

### Vaccines

According to aspects of the present disclosures, the co-vaccination syringes, systems, and/or methodologies may be used in connection with multiple types of vaccines including multiple types of delivery modalities. For example, In some embodiments, vaccines that are co-administered may include two or more of: covid-19 (i.e., Sars-Cov-2) vaccines, influenza vaccines, malaria vaccines, HIV (human immunodeficiency virus) vaccines, HSV (Herpes Simplex virus) vaccines, monkeypox vaccines, Ebola vaccines, Zika virus vaccines, RSV (Respiratory syncytial virus) vaccines, polio vaccines and/or other types of vaccines. In some embodiments, vaccines may include a delivery modality that includes lipid nanoparticles (LNPs), lipoplexes, liposomes, and/or oligosaccharide complexes and/or compounds. In some embodiments, co-administered vaccines according to the present disclosure include two different vaccines which each include the same delivery modality. In some embodiments, co-administered vaccines according to the present disclosure include two different vaccines, each including a different delivery modality.

### Methods, Devices and Systems

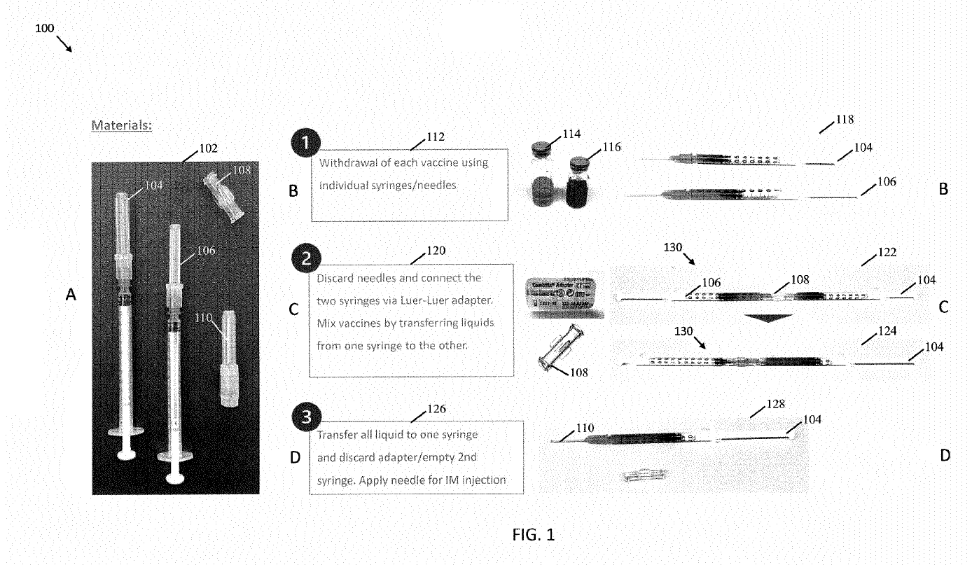

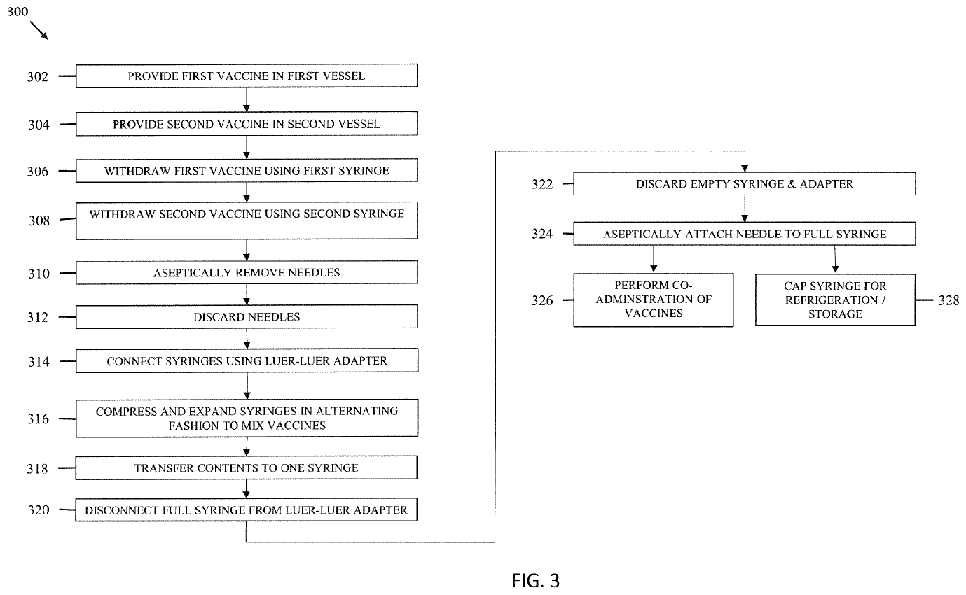

FIG. 1 depicts a schematic/workflow 100 for mixing vaccines, according to aspects of the present disclosure. As shown in panel A of FIG. 1, the materials 102 that are required for the workflow 100 include a first empty syringe 104, a second empty syringe 106, an unused needle 110, and a luer-luer adapter (or coupling) 108. The unused needle 110 is shown in FIG. 1 with a protective cap on, and in FIG. 3 without a protective cap on. In some embodiments, the luer-luer adapter 108 includes a female luer connection on each end. The workflow, as shown in panels B, C, and D of FIG. 1 (and as further described herein in connection with FIG. 3) may generally Include 3 steps including: at step 112: withdrawing each vaccine using the first and second syringes 104, 106; at step 120, removing the two needles, connecting the luer-leur adapter 108 to each of the two filled syringes 104, 106, and mixing the vaccines; and at step 126: transfering all of the liquid to one syringe, adding a new needle 110, and storing and/or administering the mixed vaccine. As shown in panel B, the first and second vaccines may be withdrwn from separate containers 114 and 116, and withdrawn into the first and second syringes 104, 106 as shown at step 118. As shown In panel C, at step 122 the workflow 100 may include linking up the first and second syringes 104, 106 via the luer-luer adapter 108 (i.e., thereby creating a syringe system 130) such that the two vaccines are separately contained within their respective first and/or second syringe 104, 106. As shown at step 124, after the two vaccines have been mixed together, the syringe system 130 includes a mixture of the vaccines (i.e., all disposed within the first syrnge 104, the plunger of which has been withdrawn. As shown at step 128, once the luer-luer adapter 108 is disconnected from both the first and second syringes 104, 106, the unused needle 110 may be connected to the first syringe 104 (i.e., for immediate administration into a patient and/or for temporary storage).

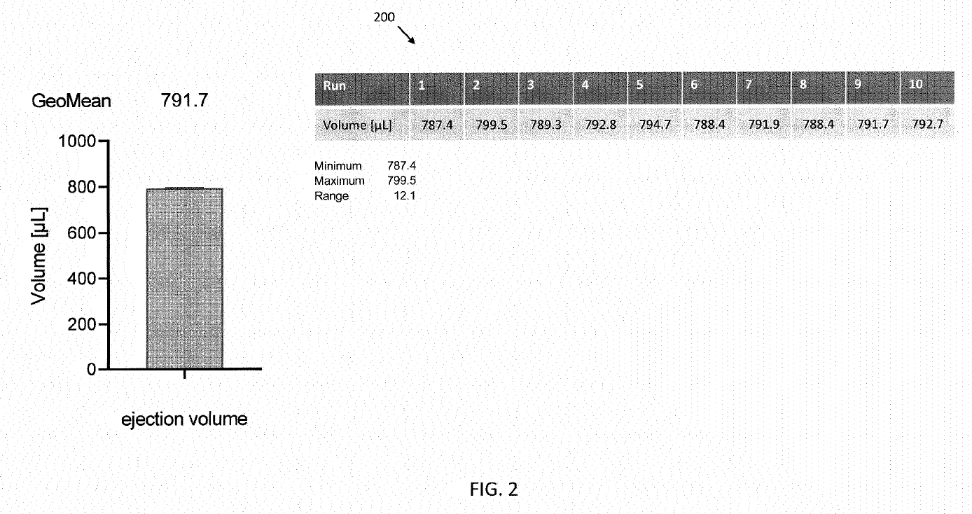

FIG. 2 illustrates data illustrating minimal loss in vaccine volume, according to aspects of the present disclosure. To generate the data 200 included in FIG. 2, vaccines were mixed using the protocol depicted in FIG. 1. The protocol was repeated n=10 times with sterile water as vaccine surrogate to determine potential volume loss (mixing 500 μL and 300 μL of sterile water, for a nominal total volume of 800 μL). Ejection volume was determined using an analytical balance with a resolution of 0.1 mg, assuming a density of water of 1 g/mL. As shown in the FIG. 2, ejection volumes observed were highly reproducible, and no relevant volume loss was observed. The ejection volumes ranged from 787.4 μL to 799.5 μL (i.e., a range of 12.1) with an average ejection volume of 791.7 μL.

FIG. 3 depicts a method 300 for mixing vaccines, according to aspects of the present disclosure. At steps 302 and 304, the method 300 may include providing first and second vaccine vessels 114, 116 (shown in FIG. 1). At steps 306 and 308, the method 300 may include using the first syringe 104 to withdraw the first vaccine from the first vessel 114, and using the second syringe 106 to withdraw the second vaccine from the second vessel 116. At step 310, the method 300 may include aseptically removing the needles from each of the first and second syringes 104, 106 without contaminating the distal ends of the syrings (which will come into contact with vaccine fluid). Accordingly, in some embodiments, aseptically removing the needles from each of the first and second syringes 104, 106 includes touching the distal end of each syringe by only touching each needle assembly to untwist each needle from the respective syringe 104, 106, and not touching any other portion of the distal ends of each syringe 104, 106. Each needle assembly may include a female luer coupling configured to interface with a male luer coupling integrated into a body of each syringe. Each of the male luer couplings may include an integrated spring valve configured to remain closed unless a pressure force in either direction exceeding a predetermined threshold is exceeded. Accordingly, the needle assembly may be removed from each syringe 104, 106 without the vaccine disposed within each syringe being discharged, as long as the plunger is not compressed.

Referring still to FIG. 3, at step 312, the method 300 may include discarding the needles (i.e., needle assemblies) once they have been removed from the first and second syringes 104, 106. At step 314, the method 300 may include connecting each of the first and second syringes 104, 106 to the luer-luer adapter 108 by inserting the male luer at the distal end of each syringe 104, 106 into the luer-luer adapter 108, and then twisting each syringe 104, 106 to secure it within the luer-luer adapter 108, and also providing a fluid-tight seal. At step 316, the method 300 may include compressing and expanding the syringes 104, 106 (i.e., using the plungers) in an alternating fashion to mix the two vaccines together. In some embodiments, compressing and expanding the syringes 104, 106 (via the plungers) in an alternating fashion includes (1) compressing the plunger of one syringe (thereby evacuating that syringe and forcing the vaccine into the other syringe, while also pushing the plunger of the other syringe outwardly), (2) compressing the plunger of the other syringe (thereby pushing the plunger of the first syringe outwardly and pushing all of the vaccine back into the first syringe), and (3) repeating the alternating compressing of plungers until the two vaccines are sufficiently mixed. In some embodiments, the two vaccines are sufficently mixed when a certain number of alternating plunger compressions are performed (for example, 1 in each direction, 2 in each direction, 3 in each direction, 4 in each direction, 5 in each direction, 6 in each direction, 7 in each direction, 8 in each direction, 9 in each direction, 10 in each direction, and/or more than 10 in each direction). In some embodiments, the two vaccines are sufficently mixed after the plunger of the first syringe 104 is compressed a single time and the original contents of both syringes are in the second syringe 106 (i.e., without compressing the plunger of the second syringe 106). In some embodiments, (for example, when there is a visible difference between the first and second vaccines after compressing the plunger of the first syringe a single time) the two vaccines are considered to be sufficently mixed when there is no discernible visible difference between the two vaccines (i.e., the two vaccines appear as a single, combined, homogenous vaccine or mixture).

Still referring to FIG. 3, at step 318, the method 300 may include transferring the full contents to one of the syringes (i.e., after sufficient mixing has been achieved). At step 320, the method 300 may include disconnecting the first syringe (i.e., the full syringe, i.e., the syringe containing the commingled vaccines) from luer-luer adapter 108. At step 322, the method 300 may include discarding the empty syringe and luer-luer adapter 108. At step 324, the method 300 may include aseptically attaching the unused needle 110 to the full syringe. At step 326, the method 300 may Include performing co-administration of the vaccines to a patient (i.e., injecting the patient using the full syringe containing the vaccine mixture). At step 328, the method 300 may include capping the full syringe (i.e., to protect the integrity of the needle 110 and to avoid the likelihood of contamination of the neelde 110) and refrigerating and/or storing the capped syringe 110. According to aspects of the present disclosure, in some embodiments, step 328 is not performed (i.e., since the combined vaccine is administered at step 326, shortly after attaching the used needle 110 at step 324 is performed). In some embodiments, step 328 (i.e., refrigerating and/or storing the full syringe containing the vaccine mixture) is performed, and then a period of time later (for example, from about 5 minutes to about 12 hours, or from about 5 minutes to about 10 hours, from about 5 minutes to about 8 hours, from about 5 minutes to about 6 hours, from about 5 minutes to about 12 hours, from about 5 minutes to about 5 hours, from about 5 minutes to about 4 hours, from about 5 minutes to about 3 hours, from about 5 minutes to about 2 hours, from about 5 minutes to about 1 hour, from about 5 minutes to about 45 minutes, from about 5 minutes to about 30 minutes, from about 5 minutes to about 20 minutes, and/or from about 5 minutes to about 15 minutes), step 326 (administering to the patient) is performed.

According to aspects of the present disclosure, the comingling of multiple different vaccines (i.e., within a single syringe) may result in various degrees of inter-vaccine interactions. Without wishing to be bound by theory, in some embodiments, commingled vaccines that include two vaccines with different delivery modalities and/or payloads may be more susceptible to inter-vaccine interactions than commingled vaccines that include the same delivery modality and/or payload. For example, in some embodiments, a protein from a first vaccine may detrimentally interfere with a lipid nanoparticle of a second vaccine, over a period of time, and vice versa. Accordingly, in some embodiments, it is preferable to administer commingled vaccines that include different delivery modalities as soon as possible after mixing (for example, within 5-10 minutes, or within 10-30 minutes, or within 0.5 hours to 1 hour, or from about 1 hour to about 3 hours, and or from about 3 hours to about 5 hours, and or from about 3 hours to about 8 hours, and/or other sub ranges therebetween). Similarly, in some embodiments, it is preferable to minimize the actual mixing of commingled vaccines that include different delivery modalities and/or payloads in order to minimize inter-vaccine interactions. Accordingly, in some embodiments according to the present disclosure, mixing the vaccines includes compressing the plunger of the first syringe 104 only a single time (thereby pushing the original contents of both syringes into the second syringe 106 (i.e., without compressing the plunger of the second syringe 106)). In some embodiments according to the present disclosure, mixing the vaccines includes compressing the plunger of the first syringe 104 gradually, for example, taking at least 1 second, at least 2 seconds, at least 3 seconds, at least 4 seconds, at least 5 seconds, at least 6 seconds, at least 7 seconds, at least 8 seconds, at least 9 seconds, at least 10 seconds, and/or more than 10 seconds to compress the plunger a single time. In some embodiments, therefore, the commingled vaccines results in a heterogeneous mixure with a first vaccine primarily occupying a distal end of the second syringe 106, and a second vaccine primarily occupying a proximal end of the second syringe 106. In some embodiments, the syringe system 130 (and/or the second 106) includes a heterogenous mixture of commingled vaccines, where at least two distinct regions of the mixture are visibly observable (i.e., visably distinguishable from one another), the two distinct regions including a first region where the first vaccine is primarily disposed and a second region where the second vaccine is primarily disposed. In some embodiments, (i.e., embodiments in which mixing the vaccines includes compressing the plunger of only a single syringe only a single time), the commingled vaccines are already disposed within a single syringe, and no further step is required to transfer the commingled vaccines to a single syringe (that is, step 318 shown in FIG. 3 does not need to be performed).

Referring to FIGS. 1 and 3, in some embodiments according the present disclosure, an alternative method of commingling and administering the vaccines includes: a) providing a first vial containing a first vaccine and a second vial containing a second vaccine, b) providing an empty syringe, c) withdrawing a dosage amount of the first vaccine from the first vial using the syringe, d) aseptically removing the needle assembly from the syringe (as described herein) and discarding the needle assembly (i.e., the original needle assembly), e) providing an unused needle/needle assembly, f) aseptically attaching the unused needle/needle assembly to the syringe, g) slowly (for example, over the course of at least 1 second, at least 2 seconds, at least 3 seconds, at least 4 seconds, at least 5 seconds, at least 7 seconds, at least 10 seconds, etc.) withdrawing a dosage amount of the second vaccine from the second vial using the syringe, thereby creating a syringe with commingled vaccines that are minimally mixed, and h) administering the commingled vaccines to a patient and/or storing the syringe containing the commingled vaccines, as described herein. Using a new needle (i.e., the unused needle) after withdrawal of the first vaccine from the first vial helps to ensure that vaccine in the second vial (which may be a multi-dose vial) is not contaminated as a result of any lingering portions of the first vaccine on the exterior of the original needle that would enter the second vial if the original needle were to be used twice.

According to aspects of the present disclosure, in some embodiments, commingled vaccines that include the same delivery modality may be mixed more thoroughly than commingled vaccines that include different delivery modalities (i.e., thereby forming a homogenous mixture). For example, in some embodiments, the mixing of commingled vaccines that include the same delivery modality may include compressing the plunger on each of the first and second syringes 104, 106 multiple times (for example, a total of 5 times each) to ensure sufficient mixing, Without wishing to be bound by theory, it may be preferable for commingled vaccines that include the same delivery modality to be more thoroughly mixed to ensure that both of the vaccines are given an approximately equal chance of being efficacious, at least in the sense of how the vaccines are delivery to, and received by, the patient. Therefore, in some embodiments, at step 316, mixing the vaccines may include: (1) compressing the plunger of only one of the syringes only a single time if the commingled vaccines include different delivery modalities, and (2) compressing the plunger on each of the first and second syringes multiple times if the commingled vaccines include the same delivery modality.

According to aspects of the present disclosure, in some embodiments, each of the two vaccines may be able to be individually stored in ambient conditions for a period of up to about 8 hours, about 12 hours, and/or up to about 24 hours. However, once commingled, the respective vaccine stabilities may decrease, especially when vaccines with two different delivery modalities are commingled. Accordingly, in some embodiments, commingled vaccines may be administered to a patience within a period of 8 hours or less, 6 hours or less, 4 hours or less, 2 hours or less, 1 hour or less, 40 minutes or less, 25 minutes or less, 15 minutes or less, 10 minutes or less, and/or 5 minutes or less.

According to aspects of the present disclosure, the combining of vaccines as described herein may be done ahead of time to reduce bottlenecks in the system (i.e., to reduce the amount of time it takes to administer multiple vaccines to a patient). The combining of vaccines ahead of time may be followed by refrigeration of the commingled vaccines, for example in expectation that a larger than normal number of patients are to receive the commingled vaccines on a given day, or within a certain timeframe.

According to aspects of the present disclosure, co-administration of vaccines may be particularly useful in connection with (1) diseases that occur in the same season and/or that are updated at similar frequencies (i.e., influenza and covid), (2) diseases that a target localized population could potentially receive in one sitting (i.e., malaria, Ebola, and/or Zika), and/or (3) diseases that a target demographic would receive in one sitting (i.e., infants, elderly people, etc.). By providing multiple vaccines simultaneously to target locations, populations, and/or demographics, time can be saved, convenience can be gained, and access to health care generally can be increased by maximizing the administrative impact (and reducing the burden) on the patience.

### Certain Exemplary Embodiments

1. A method of preparing a combination vaccine, comprising:
   (a) obtaining a first amount of a first vaccine in a first syringe,
   (b) obtaining a second amount of a second vaccine in a second syringe,
   (c) connecting the first syringe and the second syringe using an adapter,
   (d) transferring the first vaccine to the second syringe, and
   (e) discarding the first syringe.
2. The method of embodiment 1, wherein the first vaccine and the second vaccine are not stable when coformulated (e.g., wherein the immunogenicity of the first vaccine and/or the second vaccine decreases after mixing and storing at 4 °C or room temperature for 1 week or longer, 1 day or longer, or 1 hour or longer as compared to the first vaccine and/or the second vaccine stored under similar (e.g., the same) conditions).
3. The method of embodiment 1 or 2, wherein the first vaccine comprises a first delivery modality, wherein the first delivery modality comprises a lipid nanoparticle (LNP), a lipoplex (LPX), a liposome, and/or an oligosaccharide, and
   wherein the second vaccine comprises:
   (i) an inactivated virus, a recombinant polypeptide, a live attenuated virus, a subunit vaccine, or a split vaccine;
   (ii) a second delivery modality, wherein the second delivery modality comprises a lipid nanoparticle (LNP), a lipoplex, a liposome, and/or a lipophilic oligosaccharide, and wherein the second delivery modality is different from the first delivery modality; and/or
   (iii) a recombinant polypeptide.
4. The method of embodiment 3, wherein the second vaccine comprises a recombinant polypeptide comprising a lipophilic region that interacts with the first delivery modality.
5. The method of embodiment 3 or 4, wherein the second vaccine comprises a recombinant polypeptide and a lipid.
6. The method of any one of embodiments 1-5, wherein the first vaccine and the second vaccine are mixed prior to transferring the first vaccine to the second syringe, by a method comprising repeatedly transferring liquid from the first syringe to the second syringe (e.g., until the first vaccine and the second vaccine form a substantially homogenous mixture).
7. The method of any one of embodiments 1-5, wherein the first vaccine is transferred to the second syringe by a method comprising compressing the plunger of the first syringe only a single time, so that the first vaccine and the second vaccine form a substantially heterogenous mixture.
8. The method of any one of embodiments 1-7, wherein the first vaccine and/or the second vaccine are a nucleic acid vaccine.
9. The method of embodiment 8, wherein the nucleic acid vaccine is an RNA vaccine (e.g., an LNP-formulated mRNA),
10. The method of any one of embodiments 3-9, wherein the second vaccine comprises (i) one or more of an inactivated virus, a recombinant polypeptide, or a live attenuated virus; and/or (ii) comprises a non-adjuvanted vaccine, an adjuvanted vaccine, a subunit vaccine, or a split vaccine.
11. The method of any one of embodiments 1-10, wherein the first vaccine is an RNA vaccine (e.g., comprises LNP-formulated mRNA) and the second vaccine comprises an inactivated virus.
12. The method of any one of embodiments 1-10, wherein the first vaccine is an RNA vaccine (e.g., comprises LNP-formulated mRNA) and the second vaccine comprises a recombinant polypeptide (optionally with a lipophilic adjuvant).
13. The method of any one of embodiments 1-12, wherein the first vaccine and the second vaccine each deliver one or more viral antigens (e.g., wherein the first vaccine and the second vaccine each deliver one or more antigens associated with a different virus).
14. The method of embodiment 13, wherein the first vaccine and the second vaccine each deliver one or more antigens associated with a respiratory virus (e.g., wherein the first vaccine and the second vaccine each deliver one or more antigens associated with a different respiratory virus).
15. The method of any one of embodiments 1-14, wherein the first vaccine and/or the second vaccine are each a seasonally-updated vaccine (e.g., a monovalent, bivalent, trivalent, or quadrivalent seasonally-updated vaccine), optionally wherein the first vaccine and the second vaccine are updated at a similar or the same frequency.
16. The method of any one of embodiments 1-15, wherein the first vaccine is a SARS-CoV-2 vaccine.
17. The method of embodiment 16, wherein the SARS-CoV-2 vaccine is a commercially available vaccine and/or a vaccine that has been approved by a government regulatory authority (e.g., the US FDA and/or the EMA).
18. The method of embodiment 16 or 17, wherein the SARS-CoV-2 vaccine is an mRNA vaccine.
19. The method of any one of embodiments 16-18, wherein the SARS-CoV-2 vaccine comprises mRNA-1273, Ad26.CoV2.S, ChAdxOx1, NVX-CoV2373, CvnCoV, GAM-COVID0Vac, CoronaVac, BBIBP-CorV, Ad5-nCoV, zf2001, SCB-2019, JNJ 78436735, or BNT162b2.
20. The method of any one of embodiments 1-19, wherein the second vaccine is an influenza vaccine.
21. The method of embodiment 20, wherein the influenza vaccine is an inactivated influenza virus (e.g., Fluzone®, Fluzone high-dose quadrivalent®, Fluzone quadrivalent®, Fluzone Intradermal quadrivalent®, Fluzone quadrivalent southern hemisphere®, Fluad®, Fluad quadrivalent®, Afluria Quadrivalent®, Fluarix Quadrivalent®, FluLaval Quadrivalent®, or Flucelvax Quadrivalent®), a recombinant influenza vaccine (e.g., Flublok quadrivalent®), a live attenuated influenza vaccine (e.g., FluMist Quadrivalent®), an unadjuvanted influenza vaccine, an adjuvant influenza vaccine, or a subunit or split vaccine.
22. The method of any one of embodiments 1-21, wherein the second vaccine is an RSV vaccine.
23. The method of any one of embodiments 1-22, wherein the first vaccine is a SARS-CoV-2 vaccine and the second vaccine is an influenza vaccine.
24. The method of any one of embodiments 1-19 or 22, wherein the first vaccine is a SARS-CoV-2 vaccine and the second vaccine is an RSV vaccine.
25. The method of any one of embodiments 1-21 or 23, wherein the first vaccine is BNT162b2 and the second vaccine is Fluzone®, Fluzone high-dose quadrivalent®, Fluzone quadrivalent®, Fluzone intradermal quadrivalent®, or Fluzone quadrivalent southern hemisphere®.
26. The method of any one of embodiments 1-25, wherein a needle is attached to each of the first syringe and the second syringe when obtaining the first vaccine and the second vaccine, and each of the needles is removed prior to attaching the adapter.
27. The method of any one of embodiments 1-26, further comprising administering the mixture of the first vaccine and the second vaccine to a subject.
28. The method of any one of embodiments 1-27, wherein the method is performed under conditions such that there is no substantial volume loss of the first vaccine or the second vaccine (e.g., wherein the sum of the first amount of the first vaccine and the second amount of the second vaccine is approximately the same as the mixture administered to the subject, e.g., wherein the sum of the first amount of the first vaccine and the second amount of the second vaccine is within at least about 5%, at least about 4%, at least about 3%, at least about 2% or at least about 1% of the amount administered to the subject).
29. The method of any one of embodiments 1-28, wherein the method is performed in a pharmacy.
30. The method of any one of embodiments 27-29, wherein the mixture of the first vaccine and the second vaccine is administered to the subject shortly after producing the mixture (e.g., within about 1 hour, within about 30 min, within about 15 min, within about 10 min, or within about 5 min, or within about 1 min of producing the mixture).
31. The method of any one of embodiments 1-28 or 30, wherein the vaccine is administered in a hospital.
32. The method of any one of embodiments 1-31, wherein one or both of the first vaccine and the second vaccine are obtained from a multidose vial.
33. The method of any one of embodiments 1-32, wherein (i) the first amount of the first vaccine and the second amount of the second vaccine each correspond to an amount that has been shown to provide a clinical benefit and/or prophylaxis in subjects when administered alone (e.g., shown in clinical trials to provide a clinical benefit or prophylaxis), and/or (ii) the first amount of the first vaccine and the second amount of the second vaccine each correspond to a dose that has been approved for sale by a government regulatory authority (e.g., the US FDA or the EMA).
34. The method of any one of embodiments 1-33, wherein the first amount of the first vaccine and the second amount of the second vaccine are each about 100 μl to about 1 mL.
35. The method of any one of embodiments 1-34, wherein the combined volume of (a) the first amount of the first vaccine and (b) the second amount of the second vaccine, is not more than the maximum volume of the second syringe.
36. The method of any one of embodiments 1-35, wherein the combined volume of (a) the first amount of the first vaccine and (b) the second amount of the second vaccine, is not more than 1 mL.
37. The method of any one of embodiments 1-36, wherein the first amount of the first vaccine, and the second amount of the second vaccine are each about 200 μL to about 600 μL.
38. The method of embodiment 37, wherein the first amount of the first vaccine is about 500 μL and the second amount of the second vaccine is about 300 μL.
39. The method of embodiment 37, wherein the first amount of the first vaccine is about 200 μL or 300 μL.
40. The method of embodiment 37, wherein the second amount of the second vaccine is about 250 μL or about 500μL.
41. The method of embodiment 37, wherein:
   (a) the first amount of the first vaccine is about 200 μL and the second amount of the second vaccine is about 250 μL;
   (b) the first amount of the first vaccine is about 200 μL and the second amount of the second vaccine is about 500 μL;
   (c) the first amount of the first vaccine Is about 300 μL and the second amount of the second vaccine is about 250 μL; or
   (d) the first amount of the first vaccine is about 300 μL and the second amount of the second vaccine is about 500 μL.
42. The method of any one of embodiments 1-41, wherein the method further comprises:
   (f) obtaining a third amount of a third vaccine in a third syringe,
   (g) connecting the third syringe to the syringe comprising a mixture of the first vaccine and the second vaccine using an adapter,
   (h) mixing the third vaccine with the mixture of the first vaccine and the second vaccine, and
   (i) transferring the mixture of the first, second, and third vaccine to (i) the syringe that previously held the mixture of the first vaccine and the second vaccine or (ii) the third syringe, and discarding the syringe that the mixture was not transferred to.
43. The method of embodiment 42, wherein each of the first vaccine, the second vaccine, and the third vaccine deliver one or more antigens of a virus associated with a respiratory disease (e.g., wherein each vaccine delivers one or more antigens of a different virus associated with a respiratory disease).
44. The method of embodiment 42 or 43, wherein:
   (a) the first vaccine is a SARS-CoV-2 vaccine, the second vaccine is an Influenza vaccine, and the third vaccine is an RSV vaccine;
   (b) the first vaccine is a SARS-CoV-2 vaccine, the second vaccine is an RSV vaccine, and the third vaccine is an influenza vaccine;
   (c) the first vaccine is an influenza vaccine, the second vaccine is an RSV vaccine, and the third vaccine is a SARS-CoV-2 vaccine;
   (d) the first vaccine is an influenza vaccine, the second vaccine is a SARS-CoV-2 vaccine, and the third vaccine is an RSV vaccine; or
   (e) the first vaccine is a SARS-CoV-2 vaccine, the second vaccine is an RSV vaccine, and the third vaccine Is an influenza vaccine.
45. The method of any one of embodiments 1-44, wherein at least one (e.g., all) of the first syringe, the second syringe, and, if present, the third syringe, is not a uni-directional syringe (e.g., at least one of the syringes is a bidirectional syringe and/or at least one of the syringes does not have a stopper that prevents motion of the plunger in one direction).
46. The method of any one of embodiments 1-45, wherein the adapter is a sterile single-packaged adapter.
47. The method of any one of embodiments 1-46, wherein the adapter is a Luer-Luer adapter (e.g., a Luer dual female adapter).
48. A system comprising:
   a first syringe;
   an adapter comprising a first end and a second end, the first end of the adapter coupled to a distal end of the first syringe;
   a second syringe coupled at a distal end to the second end of the adapter; and
   multiple vaccines disposed within the first syringe and/or the second syringe, a first vaccine of the multiple vaccines being disposed within the first syringe and a second vaccine of the multiple vaccines being disposed within the second syringe.
49. A system comprising:
   a first syringe;
   an adapter comprising a first end and a second end, the first end of the adapter coupled to a distal end of the first syringe;
   a second syringe coupled at a distal end to the second end of the adapter;
   multiple commingled vaccines disposed within the first syringe and/or the second syringe.
50. The system of embodiment 48 or 49, wherein the first syringe and the second syringe do not comprise needles.
51. The system of any one of embodiments 48-50, wherein the commingled vaccines comprise a heterogeneous mixture with a first vaccine being primarily disposed within a distal end of one of the syringes, and a second vaccine being primarily disposed within a proximal end of the same syringe.
52. The system of embodiment 51, wherein the heterogenous mixture comprises two distinct regions, the first vaccine occupying the first distinct region and the second vaccine occupying the second distinct region,
   wherein the first and second distinct region are visibly distinguishable.
53. The system of any one of embodiments 48-50, wherein the commingled vaccines comprise a homogenous mixture.
54. A composition produced by the method of any one of embodiments 1-47.
55. A composition comprising a first vaccine and a second vaccine, wherein the first vaccine is an RNA vaccine (e.g., an mRNA-LNP vaccine) and the second vaccine is a non-nucleic acid vaccine.
56. The composition of embodiment 55, wherein the nucleic acid vaccine is an RNA vaccine.
57. The composition of embodiment 55, wherein the RNA vaccine comprises RNA formulated as particles.
58. The composition of embodiment 57, wherein the particles are lipoplex particles (LPX) or lipid nanoparticles (LNP).
59. The composition of any one of embodiments 54-58, wherein the non-nucleic acid vaccine comprises an inactivated virus, a recombinant polypeptide, a live attenuated virus, a non-adjuvanted vaccine, an adjuvanted vaccine, a subunit vaccine or a split vaccine, or any combination thereof.
60. The composition of any one of embodiments 54-59, wherein the first vaccine is an RNA vaccine (e.g., comprises LNP-formulated mRNA) and the second vaccine comprises an inactivated virus.
61. The composition of any one of embodiments 54-60, wherein the first vaccine and the second vaccine each deliver one or more viral antigens (e.g., wherein the first vaccine and the second vaccine each deliver one or more antigens associated with a different virus).
62. The composition of embodiment 61, wherein the first vaccine and the second vaccine each deliver one or more antigens associated with a respiratory virus (e.g., wherein the first vaccine and the second vaccine each deliver one or more antigens associated with a different respiratory virus).
63. The composition of any one of embodiments 54-62, wherein the first vaccine and/or the second vaccine are each a seasonally updated vaccine.
64. The composition of any one of embodiments 54-63, wherein the first vaccine is a SARS-CoV-2 vaccine.
65. The composition of embodiment 64, wherein the SARS-CoV-2 vaccine is a commercially approved vaccine.
66. The composition of embodiment 64 or 65, wherein the SARS-CoV-2 vaccine is an mRNA vaccine.
67. The composition of any one of embodiments 64-66, wherein the SARS-CoV-2 vaccine is selected from mRNA-1273, Ad26.CoV2.S, ChAdxOx1, NVX-CoV2373, CvnCoV, GAM-COVID0Vac, CoronaVac, BBIBP-CorV, Ad5-nCoV, zf2001, SCB-2019, JNJ 78436735, and BNT162b2.
68. The composition of any one of embodiments 54-67, wherein the second vaccine is an influenza vaccine.
69. The composition of embodiment 68, wherein the influenza vaccine is an inactivated influenza virus (e.g., Fluzone®, Fluzone high-dose quadrivalent®, Fluzone quadrivalent®, Fluzone intradermal quadrivalent®, Fluzone quadrivalent southern hemisphere®, Fluad®, Fluad quadrivalent®, Afluria Quadrivalent®, Fluarix Quadrivalent®, FluLaval Quadrivalent®, or Flucelvax Quadrivalent®), a recombinant influenza vaccine (e.g., Flublok quadrivalent®), a live attenuated influenza vaccine (e.g., FluMist Quadrivalent®), an unadjuvanted influenza vaccine, an adjuvant Influenza vaccine, or a subunit or split vaccine.
70. The composition of any one of embodiments 54-69, wherein the first vaccine is a SARS-CoV-2 vaccine and the second vaccine is an influenza vaccine.
71. The composition of embodiment 70, wherein the first vaccine is BNT162b2 and the second vaccine is Fluzone®, Fluzone high-dose quadrivalent®, Fluzone quadrivalent®, Fluzone intradermal quadrivalent®, or Fluzone quadrivalent southern hemisphere®.
72. The composition of any one of embodiments 54-67, wherein the second vaccine is an RSV vaccine.
73. The composition of any one of embodiments 54-67 or 72, wherein the first vaccine is a SARS-CoV-2 vaccine and the second vaccine is an RSV vaccine.
74. The composition of any one of embodiments 54-73, further comprising a third vaccine.
75. The composition of embodiment 74, wherein each of the first vaccine, the second vaccine, and the third vaccine deliver one or more antigens associated with a different virus (e.g., wherein each of the first vaccine, the second vaccine, and the third vaccine deliver an antigen associated with a different respiratory virus).
76. The composition of embodiment 74 or 75, wherein the first vaccine is a SARS-CoV-2 vaccine, the second vaccine is an influenza vaccine, and the third vaccine is an RSV vaccine.
77. The composition of any one of embodiments 54-76, wherein the first vaccine, the second vaccine, and, if present, the third vaccine, are each present in an amount that has been shown to provide a clinical benefit to subjects when administered alone (e.g., an amount shown in clinical trials to provide a clinical benefit).
78. The composition of any one of embodiments 54-77, wherein the composition comprises each of the first vaccine, the second vaccine, and if present, the third vaccine, in an amount of about 100 μl to about 1 mL.
79. The composition of any one of embodiments 54-78, wherein the combined amount of the first vaccine, the second vaccine, and, if present, the third vaccine, is not more than 1 mL.
80. The composition of any one of embodiments 54-79, wherein the combined amount of the first vaccine and the second vaccine is about 450 μL, about 550 μL, about 700 μL, about 750 μL, or about 800 μL.
81. The composition of any one of embodiments 54-80, wherein the composition is a pre-filled syringe.
82. A pre-filled syringe comprising a SARS-CoV-2 vaccine (e.g., BNT162b2).
83. A method comprising administering to a subject (i) a composition of any one of embodiments 54-81, or (ii) the contents of the pre-filled syringe of embodiment 72.
84. A method comprising administering to a subject (i) a composition of any one of embodiments 54-81, or (ii) the contents of the pre-filled syringe of embodiment 82.
85. The method of embodiment 83 or 84, wherein the method is performed in a pharmacy.
86. The method of embodiment 83 or 84, wherein the method is performed in a hospital.
87. The method of any one of embodiments 83-86, wherein the composition of any one of embodiments 54-81, or the contents of the syringe of embodiment 82, is administered via intramuscular (IM) injection.
88. The method of any one of embodiments 83-87, comprising administering a composition of embodiment 54, wherein the composition of embodiment 54 is produced shortly before being administered to the subject (e.g., produced an hour or less, 30 minutes or less, 15 minutes or less, 10 minutes or less, 5 minutes or less, or immediately before administering to a subject).
89. The method of any one of embodiments 83-88, wherein the method results in the induction of an immune response in the subject.
90. The method of any one of embodiments 83-89, wherein the method is a method of vaccinating.
89. A kit comprising (a) a first vaccine, (b) a second vaccine, and (c) instructions for performing a method of any one of embodiments 1-53.
90. The kit of embodiment 89, wherein the first vaccine and/or the second vaccine are each provided in a multi-dose vial.
91. The kit of embodiment 89, wherein the first vaccine and/or the second vaccine are each provided as a pre-filled syringe.
92. The kit of any one of embodiments 89-91, further comprising a third vaccine.
93. The kit of embodiment 92, wherein the third vaccine is provided in a multi-dose vial.
94. The kit of embodiment 92, wherein the third vaccine is provided as a prefilled syringe.
95. The kit of any one of embodiments 89-94, wherein the adapter is a sterile single-packaged adapter.
96. The kit of embodiment 95, wherein the adapter is a Luer-Luer adapter (e.g., a Luer dual female adapter).
97. The method of any one of embodiments 1-47, wherein the method produces a combination vaccine that is sterile.
98. The composition of any one of embodiments 54-81, wherein the composition is sterile.
99. A method of preparing and/or administering commingled vaccines, the method comprising:
   providing a first vial containing a first vaccine and a second vial containing a second vaccine;
   providing an empty syringe comprising a detachable needle assembly;
   withdrawing a dosage amount of the first vaccine from the first vial using the syringe;
   aseptically removing the needle assembly from the syringe;
   providing an unused needle assembly;
   aseptically attaching the unused needle assembly to the syringe; and
   slowly withdrawing a dosage amount of the second vaccine from the second vial using the syringe, thereby creating a syringe with commingled vaccines.
100. The method of embodiment 99, further comprising administering the commingled vaccines to a patient.
101. The method of embodiment 99 or 100, wherein slowly withdrawing a dosage amount of the second vaccine from the second vial comprises slowly withdrawing a dosage amount of the second vaccine from the second vial over the course of at least 5 seconds.
102. The method of any one of embodiments 99-101, wherein the first vaccine comprises a first delivery modality or payload and the second vaccine comprises a second delivery modality or payload.

### EQUIVALENTS

Certain embodiments of the present disclosure have been described above. It is, however, expressly noted that the present disclosure is not limited to those embodiments, but rather the intention is that additions and modifications to what was expressly described in the present disclosure are also included within the scope of the disclosure. Moreover, it is to be understood that the features of the various embodiments described in the present disclosure were not mutually exclusive and can exist in various combinations and permutations, even if such combinations or permutations were not made express, without departing from the spirit and scope of the disclosure. The disclosure has been described in detail with particular reference to certain embodiments thereof, but it will be understood that variations and modifications can be effected within the spirit and scope of the claimed invention.

What is claimed is:

1. A method of preparing a combination vaccine, comprising:
(a) obtaining a first amount of a first vaccine in a first syringe,
(b) obtaining a second amount of a second vaccine in a second syringe,
(c) connecting the first syringe and the second syringe using an adapter,
(d) transferring the first vaccine to the second syringe, and
(e) discarding the first syringe.

2. The method of claim 1, wherein the first vaccine and the second vaccine are not stable when coformulated (e.g., wherein the immunogenicity of the first vaccine and/or the second vaccine decreases after mixing and storing at 4 °C or room temperature for 1 week or longer, 1 day or longer, or 1 hour or longer as compared to the first vaccine and/or the second vaccine stored under similar (e.g., the same) conditions).

3. The method of claim 1 or 2, wherein the first vaccine comprises a first delivery modality, wherein the first delivery modality comprises a lipid nanoparticle (LNP), a lipoplex (LPX), a liposome, and/or an oligosaccharide, and wherein the second vaccine comprises:
(i) an inactivated virus, a recombinant polypeptide, a live attenuated virus, a subunit vaccine, or a split vaccine;
(ii) a second delivery modality, wherein the second delivery modality comprises a lipid nanoparticle (LNP), a lipoplex, a liposome, and/or a lipophilic oligosaccharide, and wherein the second delivery modality is different from the first delivery modality; and/or
(iii) a recombinant polypeptide.

4. The method of claim 3, wherein the second vaccine comprises a recombinant polypeptide comprising a lipophilic region that interacts with the first delivery modality.

5. The method of claim 3 or 4, wherein the second vaccine comprises a recombinant polypeptide and a lipid.

6. The method of any one of claim 1-5, wherein the first vaccine and the second vaccine are mixed prior to transferring the first vaccine to the second syringe, by a method comprising repeatedly transferring liquid from the first syringe to the second syringe (e.g., until the first vaccine and the second vaccine form a substantially homogenous mixture).

7. The method of any one of claims 1-5, wherein the first vaccine is transferred to the second syringe by a method comprising compressing the plunger of the first syringe only a single time, so that the first vaccine and the second vaccine form a substantially heterogenous mixture.

8. The method of any one of claims 1-7, wherein the first vaccine and/or the second vaccine are a nucleic acid vaccine,

9. The method of claim 8, wherein the nucleic acid vaccine is an RNA vaccine (e.g., an LNP-formulated mRNA).

10. The method of any one of claims 3-9, wherein the second vaccine comprises (i) one or more of an inactivated virus, a recombinant polypeptide, or a live attenuated virus; and/or (ii) comprises a non-adjuvanted vaccine, an adjuvanted vaccine, a subunit vaccine, or a split vaccine.

11. The method of any one of claim 1-10, wherein the first vaccine is an RNA vaccine (e.g., comprises LNP-formulated mRNA) and the second vaccine comprises an inactivated virus.

12. The method of any one of claim 1-10, wherein the first vaccine is an RNA vaccine (e.g., comprises LNP-formulated mRNA) and the second vaccine comprises a recombinant polypeptide (optionally with a lipophilic adjuvant).

13. The method of any one of claims 1-12, wherein the first vaccine and the second vaccine each deliver one or more viral antigens (e.g., wherein the first vaccine and the second vaccine each deliver one or more antigens associated with a different virus).

14. The method of claim 13, wherein the first vaccine and the second vaccine each deliver one or more antigens associated with a respiratory virus (e.g., wherein the first vaccine and the second vaccine each deliver one or more antigens associated with a different respiratory virus).

15. The method of any one of claims 1-14, wherein the first vaccine and/or the second vaccine are each a seasonally-updated vaccine (e.g., a monovalent, bivalent, trivalent, or quadrivalent seasonally-updated vaccine), optionally wherein the first vaccine and the second vaccine are updated at a similar or the same frequency.

16. The method of any one of claims 1-15, wherein the first vaccine is a SARS-CoV-2 vaccine.

17. The method of claim 16, wherein the SARS-CoV-2 vaccine is a commercially available vaccine and/or a vaccine that has been approved by a government regulatory authority (e.g., the US FDA and/or the EMA).

18. The method of claim 16 or 17, wherein the SARS-CoV-2 vaccine is an mRNA vaccine.

19. The method of any one of claims 16-18, wherein the SARS-CoV-2 vaccine comprises mRNA-1273, Ad26.CoV2.S, ChAdxOx1, NVX-CoV2373, CvnCoV, GAM-COVID0Vac, CoronaVac, BBIBP-CorV, Ad5-nCoV, zf2001, SCB-2019, JNJ 78436735, or BNT162b2.

20. The method of any one of claims 1-19, wherein the second vaccine is an influenza vaccine.

21. The method of claim 20, wherein the influenza vaccine is an inactivated influenza virus (e.g., Fluzone®, Fluzone high-dose quadrivalent®, Fluzone quadrivalent®, Fluzone intradermal quadrivalent®, Fluzone quadrivalent southern hemisphere®, Fluad®, Fluad quadrivalent®, Afluria Quadrivalent®, Fluarix Quadrivalent®, FluLaval Quadrivalent®, or Flucelvax Quadrivalent®), a recombinant influenza vaccine (e.g., Flublok quadrivalent®), a live attenuated influenza vaccine (e.g., FluMist Quadrivalent®), an unadjuvanted influenza vaccine, an adjuvant Influenza vaccine, or a subunit or split vaccine.

22. The method of any one of claims 1-21, wherein the second vaccine is an RSV vaccine.

23. The method of any one of claims 1-22, wherein the first vaccine is a SARS-CoV-2 vaccine and the second vaccine is an influenza vaccine.

24. The method of any one of claims 1-19 or 22, wherein the first vaccine is a SARS-CoV-2 vaccine and the second vaccine is an RSV vaccine.

25. The method of any one of claims 1-21 or 23, wherein the first vaccine is BNT162b2 and the second vaccine is Fluzone®, Fluzone high-dose quadrivalent®, Fluzone quadrivalent®, Fluzone intradermal quadrivalent®, or Fluzone quadrivalent southern hemisphere®.

26. The method of any one of claims 1-25, wherein a needle is attached to each of the first syringe and the second syringe when obtaining the first vaccine and the second vaccine, and each of the needles is removed prior to attaching the adapter.

27. The method of any one of claims 1-26, further comprising administering the mixture of the first vaccine and the second vaccine to a subject.

28. The method of any one of claims 1-27, wherein the method is performed under conditions such that there is no substantial volume loss of the first vaccine or the second vaccine (e.g., wherein the sum of the first amount of the first vaccine and the second amount of the second vaccine is approximately the same as the mixture administered to the subject, e.g., wherein the sum of the first amount of the first vaccine and the second amount of the second vaccine is within at least about 5%, at least about 4%, at least about 3%, at least about 2% or at least about 1% of the amount administered to the subject),

29. The method of any one of claims 1-28, wherein the method is performed in a pharmacy.

30. The method of any one of claims 27-29, wherein the mixture of the first vaccine and the second vaccine is administered to the subject shortly after producing the mixture (e.g., within about 1 hour, within about 30 min, within about 15 min, within about 10 min, or within about 5 min, or within about 1 min of producing the mixture).

31. The method of any one of claims 1-28 or 30, wherein the vaccine is administered in a hospital.

32. The method of any one of claims 1-31, wherein one or both of the first vaccine and the second vaccine are obtained from a multidose vial.

33. The method of any one of claims 1-32, wherein (i) the first amount of the first vaccine and the second amount of the second vaccine each correspond to an amount that has been shown to provide a clinical benefit and/or prophylaxis in subjects when administered alone (e.g., shown in clinical trials to provide a clinical benefit or prophylaxis), and/or (ii) the first amount of the first vaccine and the second amount of the second vaccine each correspond to a dose that has been approved for sale by a government regulatory authority (e.g., the US FDA or the EMA).

34. The method of any one of claims 1-33, wherein the first amount of the first vaccine and the second amount of the second vaccine are each about 100 μl to about 1 mL.

35. The method of any one of claims 1-34, wherein the combined volume of (a) the first amount of the first vaccine and (b) the second amount of the second vaccine, is not more than the maximum volume of the second syringe.

36. The method of any one of claims 1-35, wherein the combined volume of (a) the first amount of the first vaccine and (b) the second amount of the second vaccine, is not more than 1 mL.

37. The method of any one of claims 1-36, wherein the first amount of the first vaccine, and the second amount of the second vaccine are each about 200 μL to about 600 μL.

38. The method of claim 37, wherein the first amount of the first vaccine is about 500 μL and the second amount of the second vaccine is about 300 μL.

39. The method of claim 37, wherein the first amount of the first vaccine is about 200 μL or 300 μL.

40. The method of claim 37, wherein the second amount of the second vaccine is about 250 μL or about 500 μL.

41. The method of claim 37, wherein:
(a) the first amount of the first vaccine is about 200 μL and the second amount of the second vaccine is about 250 μL;
(b) the first amount of the first vaccine is about 200 μL and the second amount of the second vaccine is about 500 μL;
(c) the first amount of the first vaccine is about 300 μL and the second amount of the second vaccine is about 250 μL; or
(d) the first amount of the first vaccine is about 300 μL and the second amount of the second vaccine is about 500 μL.

42. The method of any one of claims 1-41, wherein the method further comprises:
(f) obtaining a third amount of a third vaccine in a third syringe,
(g) connecting the third syringe to the syringe comprising a mixture of the first vaccine and the second vaccine using an adapter,
(h) mixing the third vaccine with the mixture of the first vaccine and the second vaccine, and
(i) transferring the mixture of the first, second, and third vaccine to (i) the syringe that previously held the mixture of the first vaccine and the second vaccine or (ii) the third syringe, and discarding the syringe that the mixture was not transferred to.

43. The method of claim 42, wherein each of the first vaccine, the second vaccine, and the third vaccine deliver one or more antigens of a virus associated with a respiratory disease (e.g., wherein each vaccine delivers one or more antigens of a different virus associated with a respiratory disease).

44. The method of claim 42 or 43, wherein:
(a) the first vaccine is a SARS-CoV-2 vaccine, the second vaccine is an influenza vaccine, and the third vaccine is an RSV vaccine;
(b) the first vaccine is a SARS-CoV-2 vaccine, the second vaccine is an RSV vaccine, and the third vaccine is an influenza vaccine;
(c) the first vaccine is an influenza vaccine, the second vaccine is an RSV vaccine, and the third vaccine is a SARS-CoV-2 vaccine;
(d) the first vaccine is an influenza vaccine, the second vaccine is a SARS-CoV-2 vaccine, and the third vaccine is an RSV vaccine; or
(e) the first vaccine is a SARS-CoV-2 vaccine, the second vaccine is an RSV vaccine, and the third vaccine is an Influenza vaccine.

45. The method of any one of claims 1-44, wherein at least one (e.g., all) of the first syringe, the second syringe, and, if present, the third syringe, is not a uni-directional syringe (e.g., at least one of the syringes is a bidirectional syringe and/or at least one of the syringes does not have a stopper that prevents motion of the plunger in one direction).

46. The method of any one of claims 1-45, wherein the adapter is a sterile single-packaged adapter.

47. The method of any one of claims 1-46, wherein the adapter is a Luer-Luer adapter (e.g., a Luer dual female adapter).

48. A system comprising:
a first syringe;
an adapter comprising a first end and a second end, the first end of the adapter coupled to a distal end of the first syringe;
a second syringe coupled at a distal end to the second end of the adapter; and
multiple vaccines disposed within the first syringe and/or the second syringe, a first vaccine of the multiple vaccines being disposed within the first syringe and a second vaccine of the multiple vaccines being disposed within the second syringe.

49. A system comprising:
a first syringe;
an adapter comprising a first end and a second end, the first end of the adapter coupled to a distal end of the first syringe;
a second syringe coupled at a distal end to the second end of the adapter;
multiple commingled vaccines disposed within the first syringe and/or the second syringe.

50. The system of claim 48 or 49, wherein the first syringe and the second syringe do not comprise needles.

51. The system of any one of claims 48-50, wherein the commingled vaccines comprise a heterogeneous mixture with a first vaccine being primarily disposed within a distal end of one of the syringes, and a second vaccine being primarily disposed within a proximal end of the same syringe.

52. The system of claim 51, wherein the heterogenous mixture comprises two distinct regions, the first vaccine occupying the first distinct region and the second vaccine occupying the second distinct region,
wherein the first and second distinct region are visibly distinguishable.

53. The system of any one of claims 48-50, wherein the commingled vaccines comprise a homogenous mixture.

54. A composition produced by the method of any one of claims 1-47.

55. A composition comprising a first vaccine and a second vaccine, wherein the first vaccine is an RNA vaccine (e.g., an mRNA-LNP vaccine) and the second vaccine is a non-nucleic acid vaccine.

56. The composition of claim 55, wherein the nucleic acid vaccine is an RNA vaccine.

57. The composition of claim 55, wherein the RNA vaccine comprises RNA formulated as particles.

58. The composition of claim 57, wherein the particles are lipoplex particles (LPX) or lipid nanoparticles (LNP).

59. The composition of any one of claims 54-58, wherein the non-nucleic acid vaccine comprises an inactivated virus, a recombinant polypeptide, a live attenuated virus, a non-adjuvanted vaccine, an adjuvanted vaccine, a subunit vaccine or a split vaccine, or any combination thereof.

60. The composition of any one of claims 54-59, wherein the first vaccine is an RNA vaccine (e.g., comprises LNP-formulated mRNA) and the second vaccine comprises an inactivated virus.

61. The composition of any one of claims 54-60, wherein the first vaccine and the second vaccine each deliver one or more viral antigens (e.g., wherein the first vaccine and the second vaccine each deliver one or more antigens associated with a different virus).

62. The composition of claim 61, wherein the first vaccine and the second vaccine each deliver one or more antigens associated with a respiratory virus (e.g., wherein the first vaccine and the second vaccine each deliver one or more antigens associated with a different respiratory virus).

63. The composition of any one of claims 54-62, wherein the first vaccine and/or the second vaccine are each a seasonally updated vaccine.

64. The composition of any one of claims 54-63, wherein the first vaccine is a SARS-CoV-2 vaccine.

65. The composition of claim 64, wherein the SARS-CoV-2 vaccine is a commercially approved vaccine.

66. The composition of claim 64 or 65, wherein the SARS-CoV-2 vaccine is an mRNA vaccine.

67. The composition of any one of claims 64-66, wherein the SARS-CoV-2 vaccine is selected from mRNA-1273, Ad26.CoV2.S, ChAdxOx1, NVX-CoV2373, CvnCoV, GAM-COVID0Vac, CoronaVac, BBIBP-CorV, Ad5-nCoV, zf2001, SCB-2019, JNJ 78436735, and BNT162b2.

68. The composition of any one of claims 54-67, wherein the second vaccine is an influenza vaccine.

69. The composition of claim 68, wherein the influenza vaccine is an Inactivated influenza virus (e.g., Fluzone®, Fluzone high-dose quadrivalent®, Fluzone quadrivalent®, Fluzone intradermal quadrivalent®, Fluzone quadrivalent southern hemisphere®, Fluad®, Fluad quadrivalent®, Afluria Quadrivalent®, Fluarix Quadrivalent®, FluLaval Quadrivalent®, or Flucelvax Quadrivalent®), a recombinant influenza vaccine (e.g., Flublok quadrivalent®), a live attenuated influenza vaccine (e.g., FluMist Quadrivalent®), an unadjuvanted influenza vaccine, an adjuvant influenza vaccine, or a subunit or split vaccine.

70. The composition of any one of claims 54-69, wherein the first vaccine is a SARS-CoV-2 vaccine and the second vaccine is an influenza vaccine.

71. The composition of claim 70, wherein the first vaccine is BNT162b2 and the second vaccine is Fluzone®, Fluzone high-dose quadrivalent®, Fluzone quadrivalent®, Fluzone intradermal quadrivalent®, or Fluzone quadrivalent southern hemisphere®.

72. The composition of any one of claims 54-67, wherein the second vaccine is an RSV vaccine.

73. The composition of any one of claims 54-67 or 72, wherein the first vaccine is a SARS-CoV-2 vaccine and the second vaccine is an RSV vaccine.

74. The composition of any one of claims 54-73, further comprising a third vaccine.

75. The composition of claim 74, wherein each of the first vaccine, the second vaccine, and the third vaccine deliver one or more antigens associated with a different virus (e.g., wherein each of the first vaccine, the second vaccine, and the third vaccine deliver an antigen associated with a different respiratory virus).

76. The composition of claim 74 or 75, wherein the first vaccine is a SARS-CoV-2 vaccine, the second vaccine is an influenza vaccine, and the third vaccine is an RSV vaccine.

77. The composition of any one of claims 54-76, wherein the first vaccine, the second vaccine, and, if present, the third vaccine, are each present in an amount that has been shown to provide a clinical benefit to subjects when administered alone (e.g., an amount shown in clinical trials to provide a clinical benefit).

78. The composition of any one of claims 54-77, wherein the composition comprises each of the first vaccine, the second vaccine, and if present, the third vaccine, in an amount of about 100 μl to about 1 mL.

79. The composition of any one of claims 54-78, wherein the combined amount of the first vaccine, the second vaccine, and, if present, the third vaccine, is not more than 1 mL.

80. The composition of any one of claims 54-79, wherein the combined amount of the first vaccine and the second vaccine is about 450 μL, about 550 μL, about 700 μL, about 750 μL, or about 800 μL.

81. The composition of any one of claims 54-80, wherein the composition is a pre-filled syringe.

82. A pre-filled syringe comprising a SARS-CoV-2 vaccine (e.g., BNT162b2).

83. A method comprising administering to a subject (i) a composition of any one of claims 54-81, or (ii) the contents of the pre-filled syringe of claim 72.

84. A method comprising administering to a subject (i) a composition of any one of claims 54-81, or (ii) the contents of the pre-filled syringe of claim 82.

85. The method of claim 83 or 84, wherein the method is performed in a pharmacy.

86. The method of claim 83 or 74, wherein the method is performed in a hospital.

87. The method of any one of claims 83-86, wherein the composition of any one of claims 54-81, or the contents of the syringe of claim 82, is administered via intramuscular (IM) injection.

88. The method of any one of claims 83-87, comprising administering a composition of claim 54, wherein the composition of claim 54 is produced shortly before being administered to the subject (e.g., produced an hour or less, 30 minutes or less, 15 minutes or less, 10 minutes or less, 5 minutes or less, or immediately before administering to a subject).

89. The method of any one of claims 83-88, wherein the method results in the induction of an immune response in the subject.

90. The method of any one of claims 83-89, wherein the method is a method of vaccinating.

91. A kit comprising (a) a first vaccine, (b) a second vaccine, and (c) instructions for performing a method of any one of claims 1-53.

92. The kit of claim 91, wherein the first vaccine and/or the second vaccine are each provided in a multi-dose vial.

93. The kit of claim 91, wherein the first vaccine and/or the second vaccine are each provided as a pre-filled syringe.

94. The kit of any one of claims 91-93, further comprising a third vaccine.

95. The kit of claim 94, wherein the third vaccine is provided in a multi-dose vial.

96. The kit of claim 94, wherein the third vaccine is provided as a prefilled syringe.

97. The kit of any one of claims 91-96, wherein the adapter is a sterile single-packaged adapter.

98. The kit of claim 97, wherein the adapter is a Luer-Luer adapter (e.g., a Luer dual female adapter).

99. The method of any one of claims 1-47, wherein the method produces a combination vaccine that is sterile.

100. The composition of any one of claims 54-81, wherein the composition is sterile.

101. A method of preparing and/or administering commingled vaccines, the method comprising:
providing a first vial containing a first vaccine and a second vial containing a second vaccine;
providing an empty syringe comprising a detachable needle assembly;
withdrawing a dosage amount of the first vaccine from the first vial using the syringe;
aseptically removing the needle assembly from the syringe;
providing an unused needle assembly;
aseptically attaching the unused needle assembly to the syringe; and
slowly withdrawing a dosage amount of the second vaccine from the second vial using the syringe, thereby creating a syringe with commingled vaccines.

102. The method of claim 101, further comprising administering the commingled vaccines to a patient.

103. The method of claims 101 or 102, wherein slowly withdrawing a dosage amount of the second vaccine from the second vial comprises slowly withdrawing a dosage amount of the second vaccine from the second vial over the course of at least 5 seconds.

104. The method of any one of claims 101-103, wherein the first vaccine comprises a first delivery modality or payload and the second vaccine comprises a second delivery modality or payload.
